(19) United States
(12) Patent Application Publication
(10) Pub. No.: US 2023/0380674 A1
(43) Pub. Date: Nov. 30, 2023

(54) ENDOSCOPE

(71) Applicant: FUJIFILM Corporation, Tokyo (JP)

(72) Inventors: Takashi HARADA, Kanagawa (JP); Masaya INOUE, Kanagawa (JP)

(73) Assignee: FUJIFILM Corporation, Tokyo (JP)

(21) Appl. No.: 18/362,949

(22) Filed: Jul. 31, 2023

Related U.S. Application Data
(63) Continuation of application No. PCT/JP2022/004049, filed on Feb. 2, 2022.

(30) Foreign Application Priority Data
Feb. 4, 2021 (JP) 2021-016774

(51) Int. Cl. A61B 1/05; A61B 1/00

(52) U.S. Cl. CPC A61B 1/053; A61B 1/00096; A61B 1/00101; A61B 1/00103; A61B 1/00105; A61B 1/00128

(57) ABSTRACT

A distal end portion body is disposed at a distal end of an insertion part of the endoscope. In the distal end portion body, an imaging unit that is housed in an airtight or liquidtight state with respect to an external space and that is attachably and detachably held is provided. The distal end portion body includes a lens cover, an imaging unit housing portion in which the imaging unit is held, and a first opening portion and the imaging unit can be separated through the first opening portion in a case where the imaging unit held in the imaging unit housing portion is pulled to a proximal end side of the distal end portion body.

### CROSS-REFERENCE TO RELATED APPLICATIONS

This application is a Continuation of PCT International Application No. PCT/JP2022/004049 filed on 2 Feb. 2022, which claims priority under 35 U.S.C § 119(a) to Japanese Patent Application No. 2021-016774 filed on 4 Feb. 2021. The above application is hereby expressly incorporated by reference, in its entirety, into the present application.

### BACKGROUND OF THE INVENTION

### 1. Field of the Invention

The present invention relates to an endoscope.

### 2. Description of the Related Art

An endoscope used in the medical field generally includes an insertion part to be inserted into a subject and an operation part installed consecutively to a proximal end portion of the insertion part. The insertion part includes a bendable portion that is freely bendable and a distal end portion that is installed consecutively to a distal end of the bendable portion. A doctor, who is a user, can bend the bendable portion and cause the distal end portion to face a desired direction by operating an operation member provided at the operation part. In addition, it is possible to image an observation target by using an imaging unit provided at the distal end portion and to cause a monitor to display an observation image based on an imaging signal.

In addition, the endoscope is used not only for observation of the inside of the subject but also for various treatments to be performed on an observation site. Specifically, various treatment tools, such as forceps and an incision tool, are inserted through a forceps channel in the insertion part from a forceps port provided in the operation part of the endoscope, and are led out from a forceps outlet that is open at a distal end of the insertion part, whereby various treatments, such as resection and collection of the observation site, are performed (refer to JP3713286B (corresponding to U.S. Pat. No. 5,460,168A) and JP2015-178045A).

### SUMMARY OF THE INVENTION

As described above, the mechanisms of endoscopes are complicated and the endoscopes are composed of a large number of components. Therefore, many of the endoscopes are very expensive. Therefore, it is common to perform cleaning, disinfection, sterilization, and the like with respect to a used endoscope for reuse. Meanwhile, since the endoscopes are inserted into subjects, dirt such as a body fluid adheres thereto. In addition, doctors operate the endoscopes by directly touching the endoscopes. For this reason, it is desired to use a disposable endoscope in order to prevent contact between an used endoscope with dirt such as a body fluid adhering thereto and a doctor or a medical staff as far as possible and to prevent infection caused by pathogenic bacterium or viruses.

However, since an imaging unit that is built into a distal end portion of an endoscope and that is composed of an image sensor and the like is expensive, it is difficult to reduce the cost. In addition, a high-performance imaging unit is necessary after all since a high-resolution and high-quality endoscopic image is to be obtained. Therefore, an endoscope of which components other than an imaging unit are discarded and with which it is possible to obtain a high-quality and high-resolution endoscopic image, of which the resolution and the quality are equivalent to the resolution and the quality of an image obtained by means of a reusable endoscope in the related art, and to prevent infection is desired.

The present invention has been made in view of the above circumstances, and an object of the present invention is to provide a disposable endoscope of which an imaging unit can be reused easily.

The endoscope according to the present invention includes an insertion part, a distal end portion body, and an imaging unit. The insertion part is to be inserted into a subject. The distal end portion body is disposed at a distal end of the insertion part. The imaging unit is housed in the distal end portion body in an airtight or liquidtight state with respect to an external space and is attachably and detachably held.

It is preferable that the distal end portion body includes a lens cover through which at least a portion of the imaging unit is exposed, a holding portion in which the imaging unit is held, and a first opening portion that is positioned on a proximal end side of the holding portion, and the imaging unit is separated from the holding portion through the first opening portion in a case where the imaging unit held in the holding portion is pulled toward a proximal end side of the distal end portion body.

It is preferable that the holding portion includes a locking portion that locks the imaging unit and the imaging unit is allowed to move to the proximal end side in a case where the imaging unit is unlocked and it is more preferable that the locking portion is a locking claw that locks a proximal end surface of the imaging unit.

It is preferable that the distal end portion body includes a lens cover through which at least a portion of the imaging unit is exposed, a second opening portion at which the lens cover is attachably and detachably held, and a holding portion in which the imaging unit is held, and the imaging unit is separated from the holding portion through the second opening portion in a case where the lens cover held at the second opening portion is removed and the imaging unit held in the holding portion is pulled toward the second opening portion side.

It is preferable that the distal end portion body includes a rail portion that is slidably fitted to a side edge of the lens cover, and the lens cover is disposed at a position facing the imaging unit in a case where the side edge is fitted to the rail portion.

It is preferable that the distal end portion body includes a retaining member that is attachably and detachably coupled to the distal end portion body on a trajectory of the lens cover sliding with respect to the rail portion, and the retaining member restricts the lens cover from being separated in a slide direction.

It is preferable that the insertion part is composed of a distal end portion including the distal end portion body, a bendable portion installed consecutively to the distal end portion, and a soft portion installed consecutively to the bendable portion, the distal end portion body includes a lens cover through which at least a portion of the imaging unit is exposed, a holding portion in which the imaging unit is held, and an engaging portion that attachably and detachably engages with the bendable portion or a fixing member fixed to the bendable portion, and the imaging unit is separated from the bendable portion or the fixing member integrally with the holding portion in a case where the engaging portion is disengaged from the bendable portion or the fixing member.

It is preferable that a packing member that maintains airtightness or liquidtightness between the holding portion and the bendable portion or the fixing member is provided.

It is preferable that the imaging unit includes a chamfer portion that is inclined from an upper end surface facing the lens cover to a proximal end surface or from the upper end surface to a distal end surface, the distal end portion body includes an elastic body that is disposed between the lens cover and the imaging unit, and the imaging unit comes into contact with a distal end surface or a proximal end surface of the holding portion or a corner portion of the holding portion that is positioned diagonally opposite to the chamfer portion with the chamfer portion being pressed by an elastic force of the elastic body.

It is preferable that a relay connector that is coupled to the imaging unit and a cable that is connected to the imaging unit via the relay connector are provided and the relay connector is disposed inside the distal end portion body.

It is preferable that the lens cover is fixed to the distal end portion body, and the distal end portion body includes a jig cutout portion for insertion of a jig that unfixes the lens cover, the jig cutout portion being at a position where the jig cutout portion adjoins the lens cover.

It is preferable that the holding portion includes a locking portion that locks the imaging unit, the distal end portion body includes an unlocking hole that communicates with the holding portion, and the imaging unit is unlocked or at least a portion of the locking claw is removed in a case where a jig is inserted through the unlocking hole to press a locking claw and it is more preferable that the locking portion is a locking claw that locks a proximal end surface of the imaging unit.

It is preferable that the imaging unit includes a light source that irradiates a subject with illumination light. It is preferable that at least a portion of the endoscope is disposable except the imaging unit.

According to the present invention, it is possible to provide a disposable endoscope of which an imaging unit can be reused easily.

### BRIEF DESCRIPTION OF THE DRAWINGS

FIG. 1 is a schematic diagram showing a configuration of an endoscope system.

FIG. 2 is an external view of an endoscope and a treatment tool.

FIG. 3 is a perspective view showing a distal end portion of the endoscope.

FIG. 4 is an exploded perspective view of the distal end portion of the endoscope.

FIG. 5 is a plan view of a distal end portion body.

FIG. 6 is an exploded perspective view of the distal end portion body.

FIG. 7 is a main part cross-sectional view of the distal end portion of the endoscope.

FIG. 8 is a perspective view showing a step of separating an imaging unit from the distal end portion body.

FIG. 9 is a perspective view of a jig used in the step of separating the imaging unit.

FIGS. 10A and 10B are explanatory views showing a step of separating the imaging unit from the distal end portion body.

FIG. 11 is a main part cross-sectional view showing a state in which a connection piece of the jig is hooked on a connection opening portion of the imaging unit.

FIG. 12 is an exploded perspective view of a distal end portion body of an endoscope in a second embodiment.

FIG. 13 is a perspective view of an imaging unit in the second embodiment.

FIG. 14 is a main part cross-sectional view of a distal end portion of the endoscope in the second embodiment.

FIG. 15 is a perspective view showing a configuration around the imaging unit in the second embodiment.

FIG. 16 is an explanatory view showing a step of removing a lens cover from the distal end portion body of the endoscope.

FIG. 17 is a perspective view showing a modification example in which a lens cover is attachably and detachably held with respect to a distal end portion body of an endoscope.

FIG. 18 is a main part cross-sectional view of the modification example in which the lens cover is attachably and detachably held with respect to the distal end portion body of the endoscope.

FIG. 19 is an exploded perspective view showing a modification example in which a retaining member is disposed at an intermediate portion of a rail portion.

FIG. 20 is a perspective view showing the modification example in which the retaining member is disposed at the intermediate portion of the rail portion.

FIG. 21 is a perspective view of a distal end portion body and a fixing member in a third embodiment.

FIG. 22 is a perspective view of a state in which the distal end portion body and the fixing member in the third embodiment are decoupled from each other.

FIG. 23 is a perspective view of the distal end portion body in the third embodiment.

FIG. 24 is a main part cross-sectional view of the distal end portion body in the third embodiment.

FIG. 25 is a perspective view of an imaging unit in the third embodiment.

FIG. 26 is an explanatory view showing a step of unlocking the imaging unit by inserting a jig into the distal end portion body in the third embodiment.

FIG. 27 is a perspective view showing a configuration around the imaging unit in the third embodiment and is a perspective view showing a step of unlocking the imaging unit by inserting the jig.

FIG. 28 is an exploded perspective view of a distal end portion body in a fourth embodiment.

FIG. 29 is a perspective view of a portion of the distal end portion body in the fourth embodiment.

FIG. 30 is an explanatory view showing a step of unlocking an imaging unit by inserting a jig into a portion of the distal end portion body in the fourth embodiment.

FIG. 31 is a schematic diagram showing a configuration of a modification example of an endoscope system that includes a light source device.

### DESCRIPTION OF THE PREFERRED EMBODIMENTS

### First Embodiment

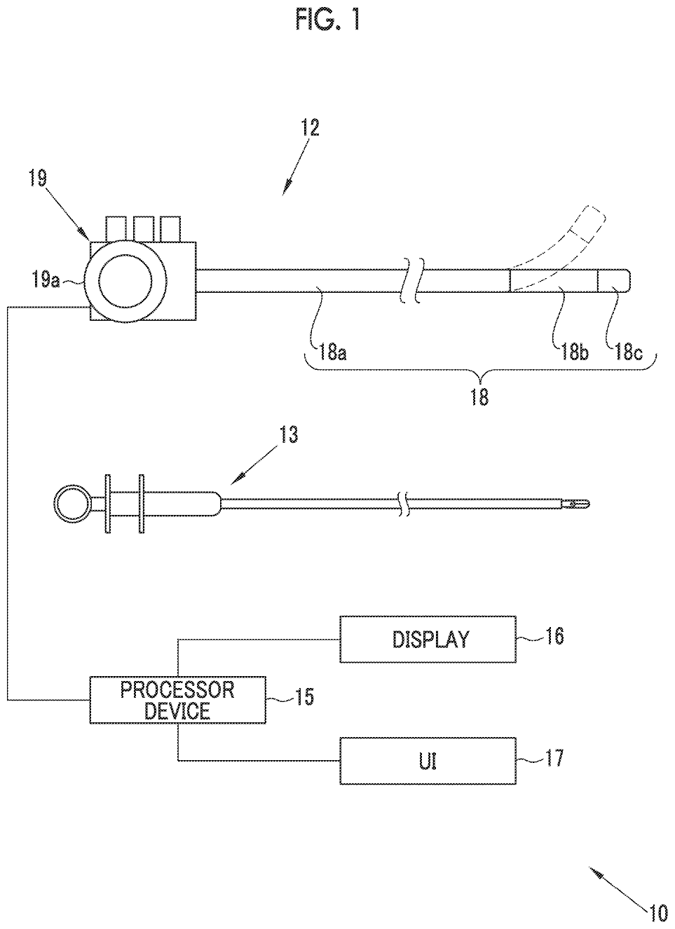

As shown in FIG. 1, an endoscope system 10 comprises an endoscope 12, a treatment tool 13, a processor device 15, a display 16, and a user interface (UI) 17. The endoscope 12 images an observation target. The processor device 15 performs system control of the endoscope system 10. The display 16 is a display unit that displays an observation image or the like based on an endoscopic image. The UI 17 includes a keyboard, a mouse, a touch pad, a microphone, and the like, and receives an input operation of a doctor who is a user.

The endoscope 12 is electrically connected to the processor device 15. The endoscope 12 has an insertion part 18 to be inserted into a subject and an operation part 19 provided at a proximal end portion of the insertion part 18. The insertion part 18 includes a soft portion 18a, a bendable portion 18b, and a distal end portion 18c which are consecutively installed in this order from the proximal end toward a distal end. The bendable portion 18b is bent as an angle knob 19a of the operation part 19 is operated. As a result, the distal end portion 18c is caused to face a desired direction.

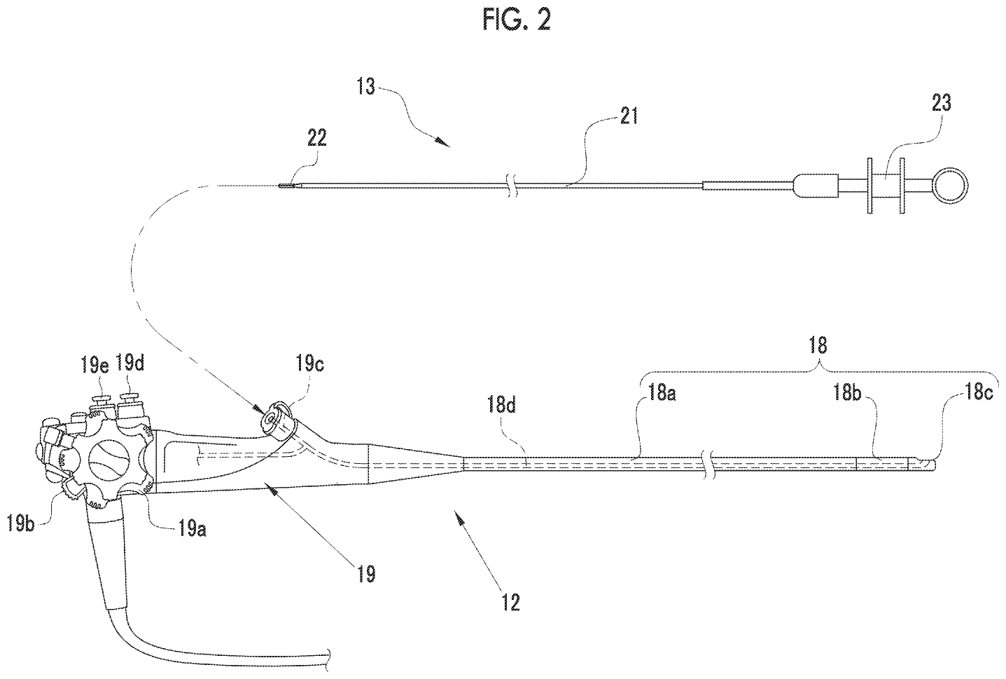

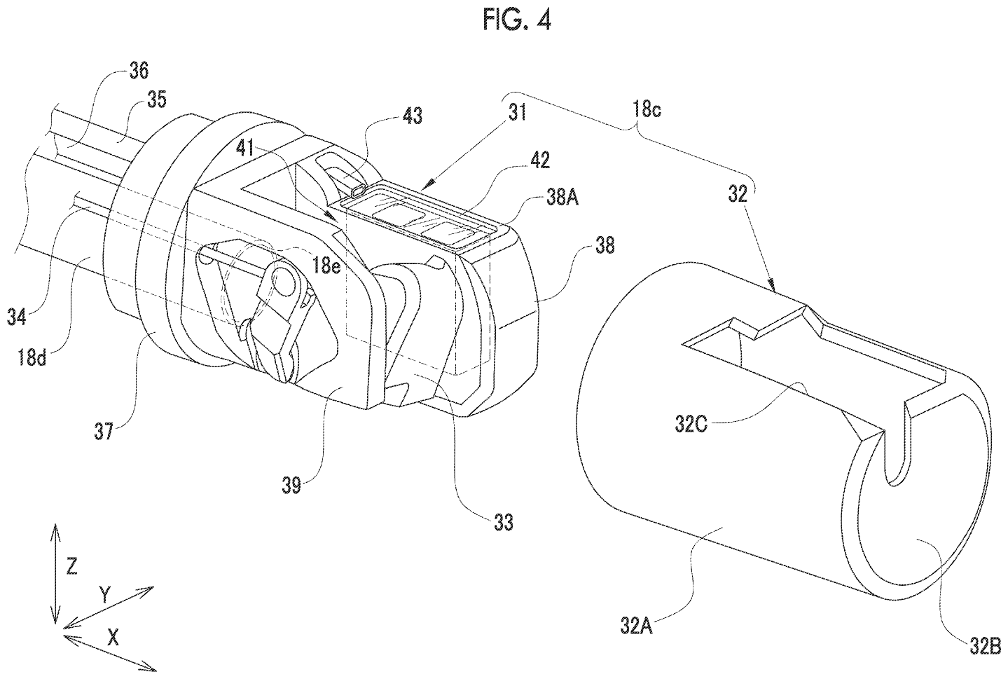

As shown in FIG. 2, the operation part 19 is provided with an elevating operation lever 19b, a treatment tool lead-in port 19c, an air and water supply button 19d, and a suction button 19e, in addition to the angle knob 19a. The treatment tool lead-in port 19c is an entrance for inserting the treatment tool 13. The treatment tool 13 inserted into the treatment tool lead-in port 19c is guided to an elevator housing portion 41 (see FIG. 4) of the distal end portion 18c.

As the elevating operation lever 19b is operated, an elevator 33 which will be described later rotates. In a case where the elevator rotates, an advancing direction of the treatment tool 13 guided to the elevator housing portion 41 is bent, so that the treatment tool 13 is guided in a direction toward an opening window 32C on an upper surface side of the elevator housing portion 41 and is led out of the opening window 32C.

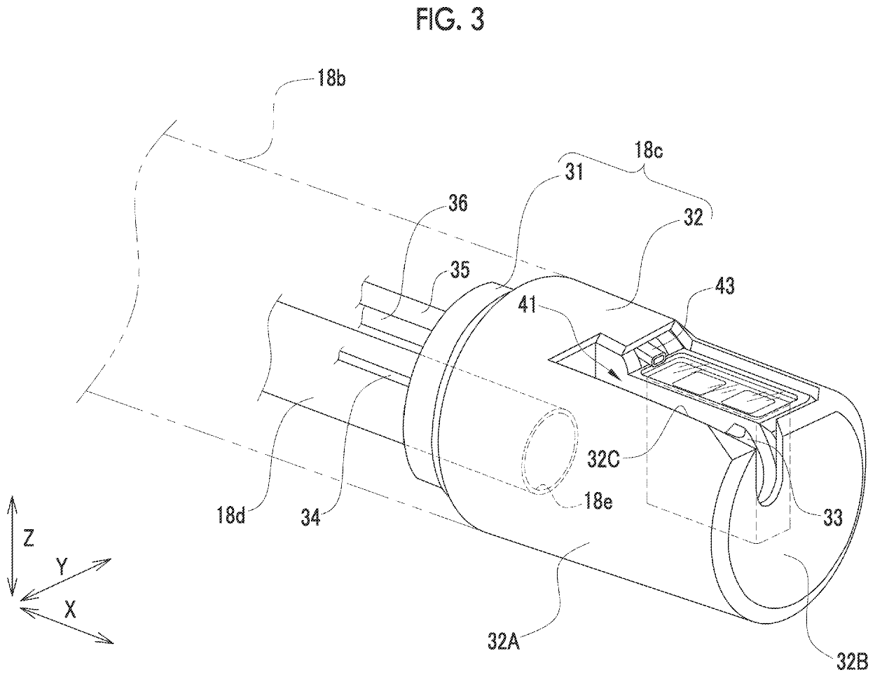

In a case where the air and water supply button 19d is operated, air and water are supplied to an air and water supply tube (not shown) and the air and water are jetted from an air and water supply nozzle 43 (see FIGS. 3 and 4) provided in a distal end portion body 31. In addition, in a case where the suction button 19e is operated, body fluids, such as blood, can be suctioned via a treatment tool channel 18d from a suction port that also serves as a treatment tool lead-out port 18e (see FIG. 4) disposed in the distal end portion body 31.

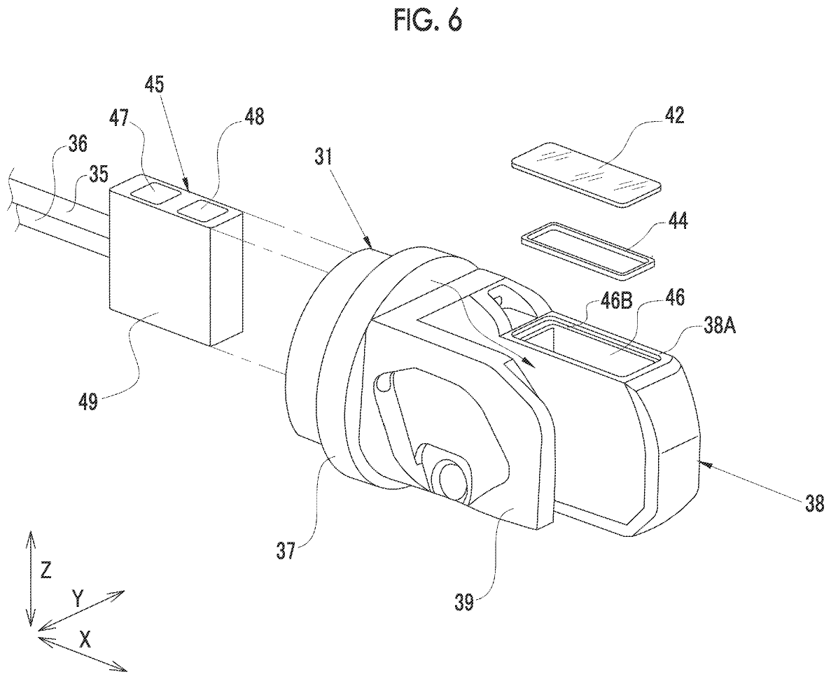

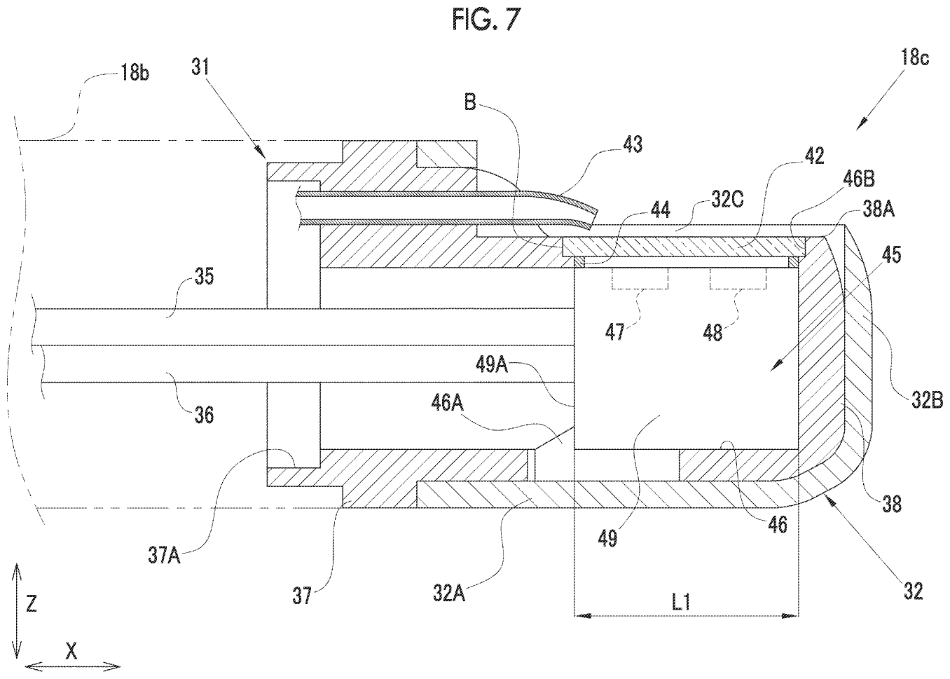

An imaging unit 45 (see FIGS. 6 and 7) which will be described later is provided at the distal end portion 18c. The imaging unit 45 is provided with an image sensor 47, a light source 48, and the like. The image sensor 47 is preferably a charge coupled device (CCD) image sensor, a complementary metal-oxide semiconductor (CMOS) image sensor, or the like. The light source 48 is preferably composed of a light emitting element such as a laser diode (LD) or a light emitting diode (LED).

The processor device 15 is electrically connected to the display 16 and the UI 17. The processor device 15 performs image processing or the like on an endoscopic image captured by the image sensor 47 and displays the image-processed endoscopic image on the display 16.

The treatment tool channel 18d for inserting the treatment tool 13 is disposed in the insertion part 18. One end of the treatment tool channel 18d is connected to the distal end portion body 31, and the other end of the treatment tool channel 18d is connected to the treatment tool lead-in port 19c provided in the operation part 19.

The treatment tool 13 is a treatment tool for an endoscope that is inserted into a subject together with the insertion part 18 through the treatment tool channel 18d. As the treatment tool 13, for example, a biopsy forcep, a snare, a high-frequency treatment tool, an ultrasonic treatment tool, and the like is combined with the endoscope 12.

The treatment tool 13 comprises a flexible sheath 21, an operation wire (not shown), a distal end portion 22, and an operation part 23. The flexible sheath 21 is a tubular sheath formed of a flexible material, for example, a soft resin, and is inserted into the treatment tool channel 18d of the endoscope 12. The operation wire is provided integrally with the distal end portion 22, and is inserted into the flexible sheath 21.

As shown in FIGS. 3 and 4, the distal end portion 18c includes the distal end portion body 31 and a cap 32, and is formed by mounting the cap 32 on the distal end portion body 31. A configuration of the distal end portion 18c is not limited thereto, and the distal end portion body 31 and the cap 32 may be integrally fixed to each other so as not to be detached by the user. The distal end portion body 31 is provided on a distal end side of the insertion part 18 (see FIG. 1), and the elevator 33 is provided in the distal end portion body 31.

The endoscope 12 is, for example, a side-viewing endoscope used as a duodenoscope, and the distal end portion 18c shown in FIGS. 3 and 4 has a configuration of the side-viewing endoscope. FIGS. 3 and 4 show the treatment tool channel 18d, an elevating operation wire 34, a signal cable 35, and an electric cable 36 which are disposed inside the insertion part 18 of the endoscope 12. The treatment tool channel 18d guides a distal end portion of the treatment tool 13 to the distal end portion body 31. The elevating operation wire 34 is an operation wire for performing an operation of rotating the elevator 33. Illustrations of an air and water supply channel and the like connected to the air and water supply nozzle 43 are omitted to prevent complication.

The cap 32 is formed in a substantially tubular shape of which a distal end side is sealed, and has a peripheral surface portion 32A and an end surface portion 32B. The opening window 32C having a substantially rectangular shape is formed in a part of the peripheral surface portion 32A. In the example shown in FIGS. 3 and 4, the opening window 32C is an opening portion cut out from the peripheral surface portion 32A to the end surface portion 32B. Hereinafter, descriptions will be made with a central axis direction of the distal end portion body 31 and the cap 32 as an X-axis direction, a vertical direction orthogonal to the X-axis direction as a Z-axis direction, and a horizontal direction orthogonal to the X-axis direction and the Z-axis direction as a Y-axis direction.

In a case where the cap 32 is mounted on the distal end portion body 31, the cap 32 covers the elevator housing portion 41 to be described later, and the opening window 32C is opened toward the Z-axis direction. Accordingly, the treatment tool lead-out port 18e of the treatment tool channel 18d communicates with the opening window 32C through the elevator housing portion 41. The cap 32 is coaxially mounted on the distal end portion body 31.

The cap 32 is made of an elastic material, for example, a rubber material such as fluororubber or silicon rubber, or a resin material such as polysulfone or polycarbonate. A protruding engaging portion (not shown) to be engaged with a groove-like engaged portion (not shown) formed on the distal end portion body 31 is provided on a proximal end side of the cap 32, and the engaging portion is engaged with the engaged portion, so that the cap 32 is attachably and detachably mounted on the distal end portion body 31.

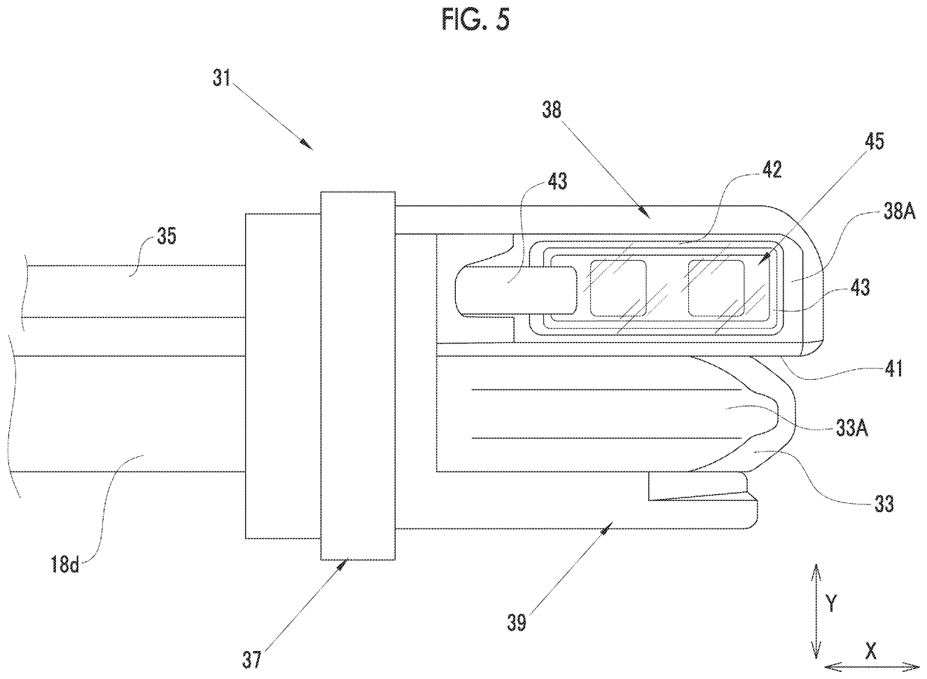

As shown in FIG. 5, the distal end portion body 31 includes a disk portion 37 and a pair of partition wall portions 38 and 39. The distal end portion body 31 is made of, for example, a resin material. The pair of partition wall portions 38 and 39 are provided to protrude from the disk portion 37 in the X-axis direction. The partition wall portions 38 and 39 are disposed opposite each other in the Y-axis direction. In addition, the elevator housing portion 41 housing the elevator 33 is provided between the partition wall portion 38 and the partition wall portion 39. The elevator housing portion 41 is opened in the Z-axis direction.

The elevator housing portion 41 communicates with the treatment tool lead-out port 18e of the treatment tool channel 18d. The elevator 33 is attached to be rotatable in the elevator housing portion 41. As the elevating operation lever 19b is operated, the elevating operation wire 34 is pulled and the elevator 33 rotates. As the elevator 33 rotates, a lead-out direction of the distal end portion 22 of the treatment tool 13 led out to the treatment tool lead-out port 18e can be changed.

The disk portion 37 is coupled to a distal end side of the bendable portion 18b. The bendable portion 18b is configured by covering an outer periphery of a structure in which a plurality of bendable pieces are rotatably connected with a tubular net body, a rubber outer skin, or the like. The disk portion 37 is fixed to the bendable piece positioned on the most distal end side among the plurality of bendable pieces forming the bendable portion 18b by, for example, screwing or adhesion using an adhesive.

The partition wall portion 38 is disposed adjacent to the elevator housing portion 41 in the Y-axis direction. The partition wall portion 38 includes a lens cover 42, the air and water supply nozzle 43, packing 44 (see FIG. 6), and the imaging unit 45. Regarding the endoscope 12 in the present embodiment, substantially all of components thereof other than the imaging unit 45 are formed of a resin material, a rubber material, a metal material, or the like and are discarded as disposables. In addition, in the case of the metal material, it is more preferable that the components are formed by metal injection molding.

The imaging unit 45 is composed of the image sensor 47, the light source 48, the signal cable 35, the electric cable 36, and a case member 49. The image sensor 47 is electrically connected to the signal cable 35, and the light source 48 is electrically connected to the electric cable 36. The case member 49 is formed in a substantially quadrangular columnar shape. The image sensor 47 and the light source 48 are exposed through an upper end surface of the case member 49 and are provided at positions where the image sensor 47 and the light source 48 face the lens cover 42 in a case where the case member 49 is housed in an imaging unit housing chamber 46. Accordingly, at least a portion of the imaging unit 45 is exposed through the lens cover 42.

The signal cable 35 and the electric cable 36 protrude from a proximal end surface of the case member 49 and are respectively connected to the processor device 15 through the insertion part 18, the operation part 19, a connector (not shown), and the like. The processor device 15 performs image processing or the like on the imaging signal acquired by the image sensor 47 and causes the display 16 to display the observation image. Through the electric cable 36, electricity is transmitted from the processor device 15 to the light source 48. The light source 48 emits light and irradiates the observation target with illumination light as the light source 48 receives power supplied thereto.

As shown in FIG. 6, the lens cover 42 is disposed on an upper end surface 38A of the partition wall portion 38 in the Z-axis direction. The imaging unit housing chamber 46 (a holding portion) is provided in the partition wall portion 38. The imaging unit housing chamber 46 is formed in a substantially quadrangular tubular shape of which a distal end side is sealed. In a cross section orthogonal to the X-axis direction, an inner peripheral surface of the imaging unit housing chamber 46 is formed to match the outer shape of the case member 49. Accordingly, in a case where the case member 49 is housed in the imaging unit housing chamber 46, the position of the case member 49 is restricted in the Z-axis direction and the Y-axis direction.

As shown in FIG. 7, the disk portion 37 includes a first opening portion 37A. The first opening portion 37A is positioned on a proximal end side of the imaging unit housing chamber 46. The first opening portion 37A is an opening portion that is cut out from an proximal end surface of the disk portion 37 and of which the opening area is larger than that of the imaging unit housing chamber 46, and the first opening portion 37A communicates with the imaging unit housing chamber 46. Accordingly, in a case where the case member 49 of the imaging unit 45 is to be housed in the imaging unit housing chamber 46 from the external space and in a case where the case member 49 is to be separated from the imaging unit housing chamber 46 and moved to the external space, the case member 49 can be moved through the first opening portion 37A.

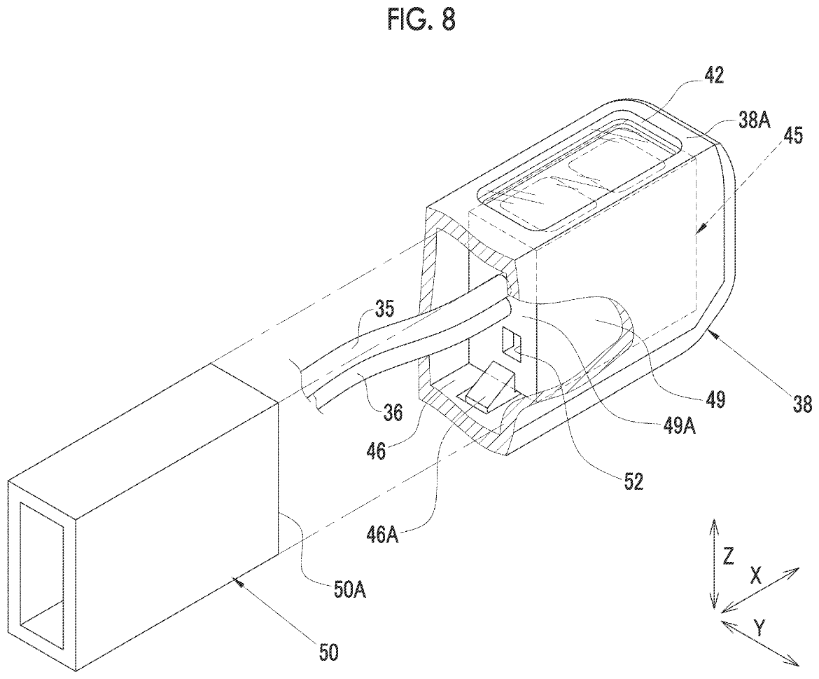

In the imaging unit housing chamber 46, a locking claw 46A (see FIG. 8) as a locking portion is formed. The locking claw 46A is disposed to be separated from a distal end of the imaging unit housing chamber 46 by an interval corresponding to a long side L1 (the length in the X-axis direction) of the case member 49. Since the case member 49 is locked by the locking claw 46A, the position of the case member 49 is restricted in the X-axis direction in a case where the case member 49 is housed in the imaging unit housing chamber 46. As described above, the case member 49 of which the position is restricted by the inner peripheral surface of the imaging unit housing chamber 46 and the locking claw 46A is attachably and detachably held in the imaging unit housing chamber 46. A locking portion locking the imaging unit 45 is not limited to the locking claw 46A as described above as long as the movement of the imaging unit 45 is restricted by the locking portion. For example, an opening portion may be formed in the imaging unit housing chamber 46 and the imaging unit 45 may be locked by a locking pin fitted to the opening portion.

In a case where the case member 49 of the imaging unit 45 is to be housed in the imaging unit housing chamber 46, the case member 49 is pushed into the imaging unit housing chamber 46 through the first opening portion 37A such that a distal end surface of the case member 49 is inserted first, for example. It is possible to cause the case member 49 to approach the distal end side of the imaging unit housing chamber 46 beyond the locking claw 46A, by pushing the case member 49. At this time, the locking claw 46A is elastically deformed by being pressed by the case member 49 and is bent toward the outside of the imaging unit housing chamber 46. Therefore, the locking claw 46A does not hinder the movement of the case member 49. In a case where the case member 49 climbs over the locking claw 46A, the locking claw 46A restores the original shape thereof because of the elastic force thereof and locks a proximal end surface 49A of the case member 49. Accordingly, the case member 49 is held in the imaging unit housing chamber 46.

A lens cover opening portion 46B is formed at an upper portion of the imaging unit housing chamber 46. The lens cover opening portion 46B is an opening portion that is cut out from the upper end surface 38A of the partition wall portion 38 in the Z-axis direction to an upper surface of the imaging unit housing chamber 46 and of which the cross section is quadrangular.

The lens cover 42 is formed of transparent glass or a resin material and is formed in a rectangular plate-like shape. The lens cover 42 is fitted to the lens cover opening portion 46B and is fixed to the partition wall portion 38 by adhesion using an adhesive or the like. Therefore, the imaging unit housing chamber 46 houses the case member 49 of the imaging unit 45 with the case member 49 being at a position facing the lens cover 42. The image sensor 47 provided in the imaging unit 45 can observe, via the lens cover 42, an upper visual field region in the Z-axis direction at which the opening window 32C is open.

In addition, the packing 44 is sandwiched between the lens cover 42 and the case member 49. The packing 44 is an elastic member that is formed of rubber or a soft resin, is slightly smaller than the lens cover 42, and has a quadrangular frame-like shape matching the inner peripheral surface of the imaging unit housing chamber 46. Therefore, in a state where the packing 44 is sandwiched between the lens cover 42 and the case member 49, the packing 44 comes into close contact with the upper end surface of the case member 49 and the inner peripheral surface of the imaging unit housing chamber 46. Accordingly, the imaging unit 45 is housed in the partition wall portion 38 in an airtight or liquidtight state with respect to the external space.

A space between the lens cover 42 and the case member 49 may be filled with a sealing material instead of the packing 44. In this case, the sealing material is preferably formed of transparent rubber or resin.

The above-described air and water supply nozzle 43 is provided at the distal end portion body 31 to face the lens cover 42. Accordingly, the lens cover 42 is cleaned by air and water jetted from the air and water supply nozzle 43. The air and water supply nozzle 43 is disposed over a boundary B (see FIG. 7) between the lens cover 42 and the lens cover opening portion 46B. That is, a distal end of the air and water supply nozzle 43 is positioned inside the lens cover 42. Accordingly, air and water jetted from the air and water supply nozzle 43 are not blocked at the boundary B and thus the lens cover 42 can be efficiently cleaned.

A step of separating the imaging unit 45 from the imaging unit housing chamber 46 after use of the endoscope 12 will be described with reference to FIGS. 8 to 11. As shown in FIG. 8, a jig 50 is used in a step of separating the imaging unit 45 from the imaging unit housing chamber 46.

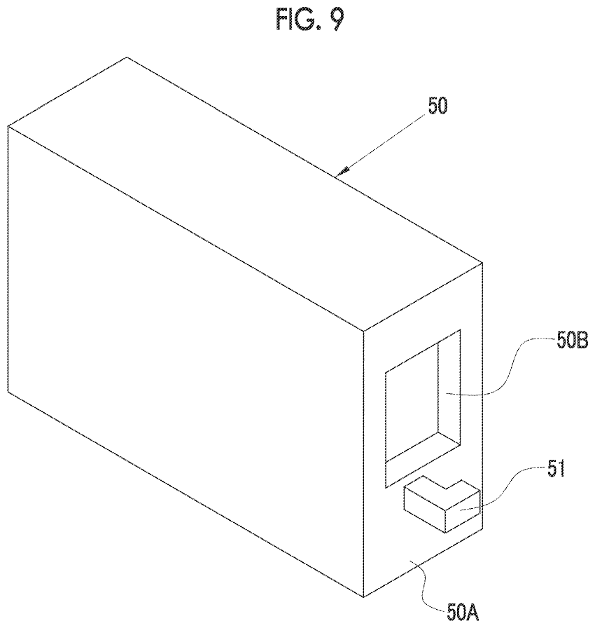

As shown in FIG. 9, the jig 50 is a jig having a quadrangular tubular shape matching the outer shape of the imaging unit 45 and a distal end surface 50A thereof is provided with a connection piece 51. The connection piece 51 is formed in an L-like shape that is bent at a substantially right angle. Meanwhile, a connection opening portion 52 (see FIG. 8) is formed in the proximal end surface 49A of the case member 49. The connection piece 51 can be inserted into the connection opening portion 52 and the connection piece 51 can be hooked on a peripheral edge of the connection opening portion 52. In addition, a through-hole 50B, through which the signal cable 35 and the electric cable 36 pass, is formed in the jig 50.

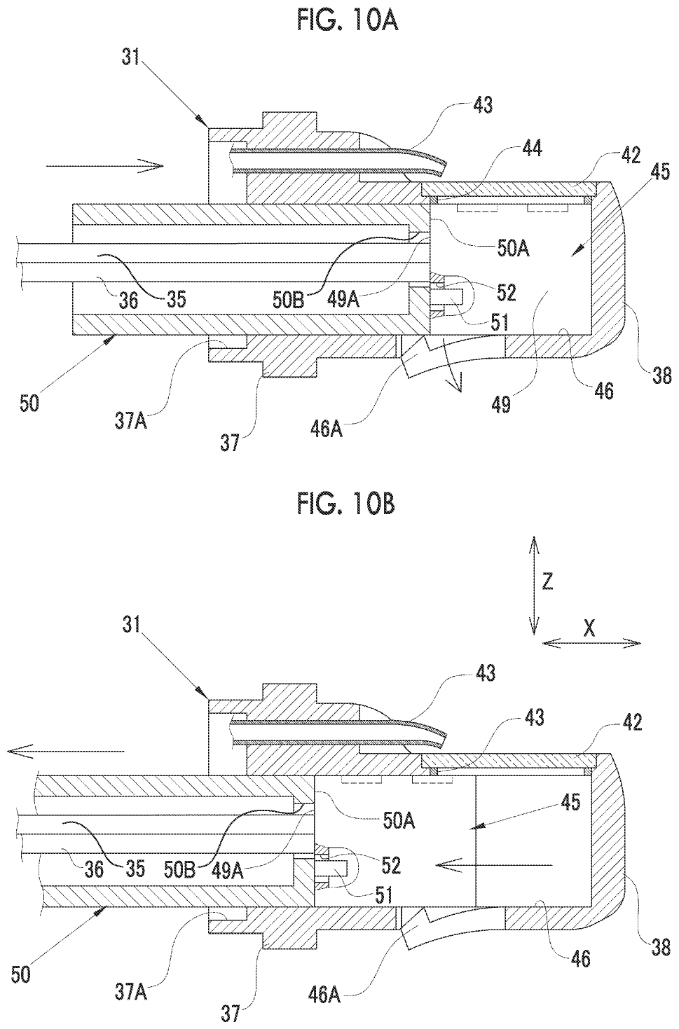

As shown in FIG. 10A, in a step of separating the imaging unit 45 from the imaging unit housing chamber 46, first, the jig 50 is inserted through the first opening portion 37A that is positioned on a proximal end side of the distal end portion body 31. In addition, at this time, the signal cable 35 and the electric cable 36 are caused to pass through the through-hole 50B in advance so as not to be sandwiched between the jig 50 and the imaging unit 45. Since the jig 50 is formed to match the outer shape of the imaging unit 45, the distal end surface 50A of the jig 50 can be caused to approach the proximal end surface 49A of the case member 49 beyond the locking claw 46A. At this time, the locking claw 46A is elastically deformed by being pressed by the jig 50 and is bent toward the outside of the imaging unit housing chamber 46. Accordingly, the locking claw 46A allows the jig 50 and the imaging unit 45 to move.

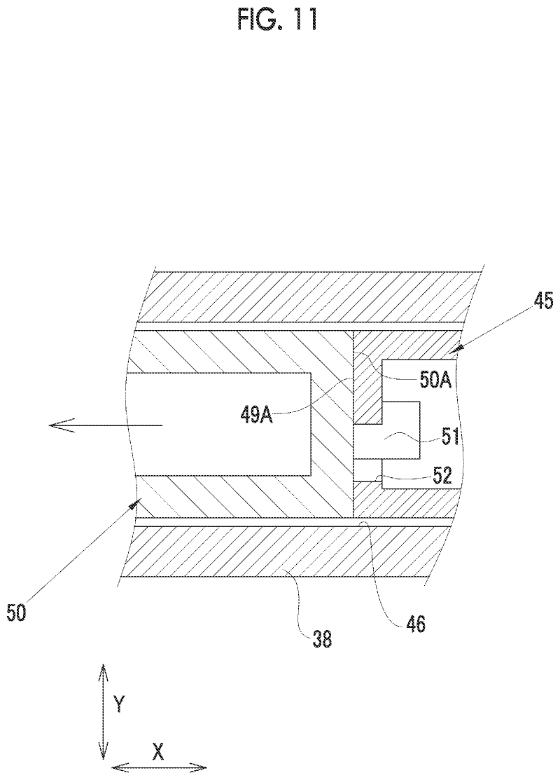

By causing the distal end surface 50A of the jig 50 to approach the proximal end surface 49A of the case member 49, it is possible to hook the connection piece 51 on the peripheral edge of the connection opening portion 52 as shown in FIGS. 10B and 11. In addition, by pulling the jig 50, it is possible to pull the imaging unit 45 to a proximal end side in the X-axis direction and to separate the imaging unit 45 from the imaging unit housing chamber 46.

As described above, the imaging unit 45 is housed and attachably and detachably held in the distal end portion body 31 in an airtight or liquidtight state with respect to the external space. Therefore, it is possible to easily remove and reuse the imaging unit 45 after use of the endoscope 12 and components of the endoscope 12 other than the imaging unit 45 can be made disposable. In addition, since the reused imaging unit 45 is housed in the distal end portion body 31 in the airtight or liquidtight state with respect to the external space, infection caused by a virus or the like can be prevented.

In addition, the distal end portion body 31 includes the lens cover 42, the imaging unit housing chamber 46 disposed at a position facing the lens cover 42, and the first opening portion 37A positioned on the proximal end side of the imaging unit housing chamber 46 and it is possible to separate the imaging unit 45 from the imaging unit housing chamber 46 by pulling the imaging unit 45 to the proximal end side. Accordingly, a step of removing the lens cover 42 is not necessary. Furthermore, it is possible to easily reuse the imaging unit 45 since removal of the imaging unit 45 is easy.

### Second Embodiment

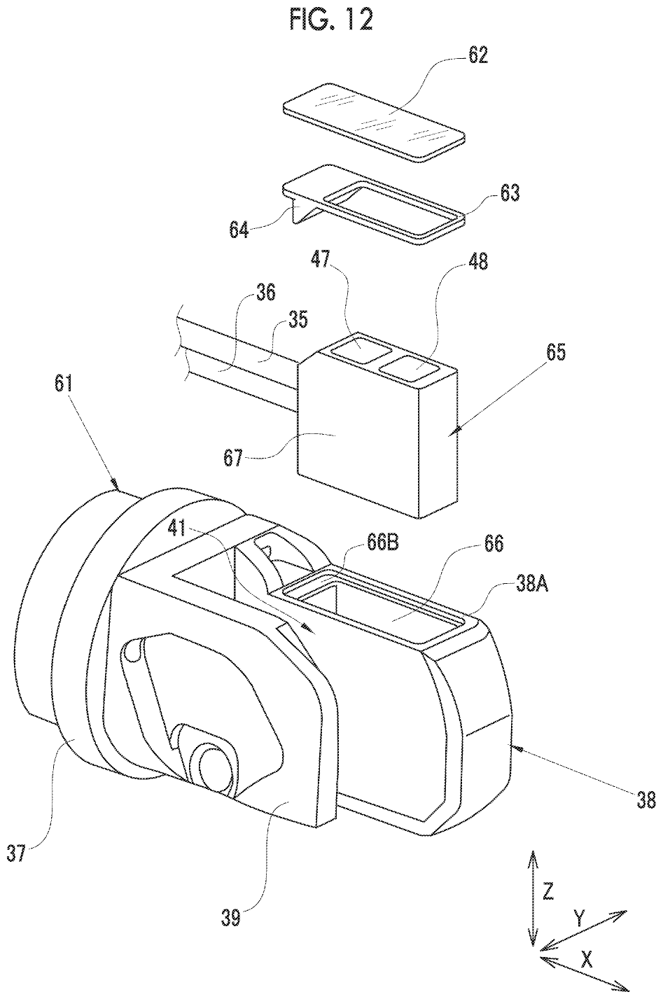

In the first embodiment described above, the first opening portion 37A positioned on the proximal end side of the imaging unit housing chamber 46 is provided and the imaging unit 45 is pulled toward the proximal end side to be separated from the imaging unit housing chamber 46. However, the present invention is not limited thereto and in a second embodiment which will be described below, an imaging unit is separated from a lens cover side. A distal end portion body 61 shown in FIG. 12 includes a lens cover 62, packing 63, a pressing member 64, and an imaging unit 65. The distal end portion 18c of the endoscope is configured by mounting the cap 32 to the distal end portion body 61.

Regarding the endoscope in the present embodiment, as with the endoscope 12 of the first embodiment, it is preferable that substantially all of components thereof other than the imaging unit 65 are formed of a resin material, a rubber material, a metal material, or the like and are discarded as disposables. In addition, it is preferable that the same applies to the embodiments below. The same components and members as those of the distal end portion 18c of the first embodiment are denoted by the same reference numerals and the descriptions thereof will be omitted.

The distal end portion body 61 includes the disk portion 37 and the pair of partition wall portions 38 and 39 as with the distal end portion body 31 of the first embodiment. The distal end portion body 61 is made of, for example, an elastic resin material. In addition, the above-described elevator housing portion 41 housing the elevator 33 is provided between the partition wall portion 38 and the partition wall portion 39.

An imaging unit housing chamber 66 (a holding portion) is provided in the partition wall portion 38. The imaging unit housing chamber 66 is formed in a substantially quadrangular tubular shape of which a distal end side is sealed. A lens cover opening portion 66B (a second opening portion) is formed at an upper portion of the imaging unit housing chamber 66. The lens cover opening portion 66B is an opening portion that is cut out from the upper end surface 38A of the partition wall portion 38 in the Z-axis direction to an upper surface of the imaging unit housing chamber 66 and of which the cross section is quadrangular.

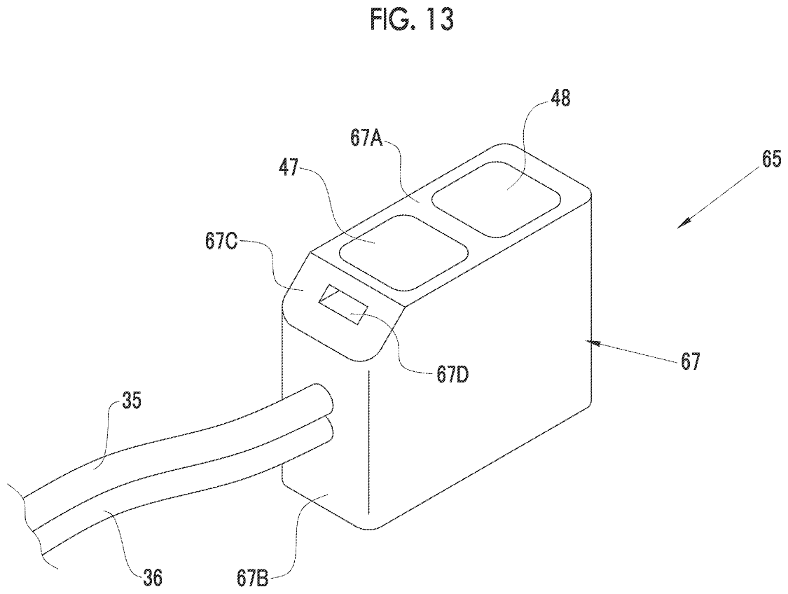

As shown in FIG. 13, the imaging unit 65 is composed of the image sensor 47, the light source 48, the signal cable 35, the electric cable 36, and a case member 67. The case member 67 is formed in a substantially quadrangular columnar shape. A chamfer portion 67C that is inclined from an upper end surface 67A to a proximal end surface 67B is formed at the case member 67. The present invention is not limited thereto and the chamfer portion 67C may be inclined from the upper end surface 67A to a distal end surface of the case member 67. In addition, a jig cutout portion 67D obtained by cutting out a portion of the chamfer portion 67C is formed in the case member 67.

The image sensor 47 and the light source 48 are exposed through the upper end surface 67A of the case member 67 and are provided at positions where the image sensor 47 and the light source 48 face the lens cover 62 in a case where the case member 67 is housed in an imaging unit housing chamber 66. Accordingly, at least a portion of the imaging unit 65 is exposed through the lens cover 62.

As with the lens cover 42 of the first embodiment, the lens cover 62 is formed of transparent glass or a resin material and is formed in a rectangular plate-like shape. Although details will be described later, the lens cover 62 is held to be attachable and detachable with respect to the lens cover opening portion 66B. The imaging unit housing chamber 66 houses the case member 67 of the imaging unit 65 with the case member 67 being at a position facing the lens cover 62. The image sensor 47 provided in the imaging unit 65 can observe, via the lens cover 62 and the pressing member 64, an upper visual field region in the Z-axis direction at which the opening window 32C is open.

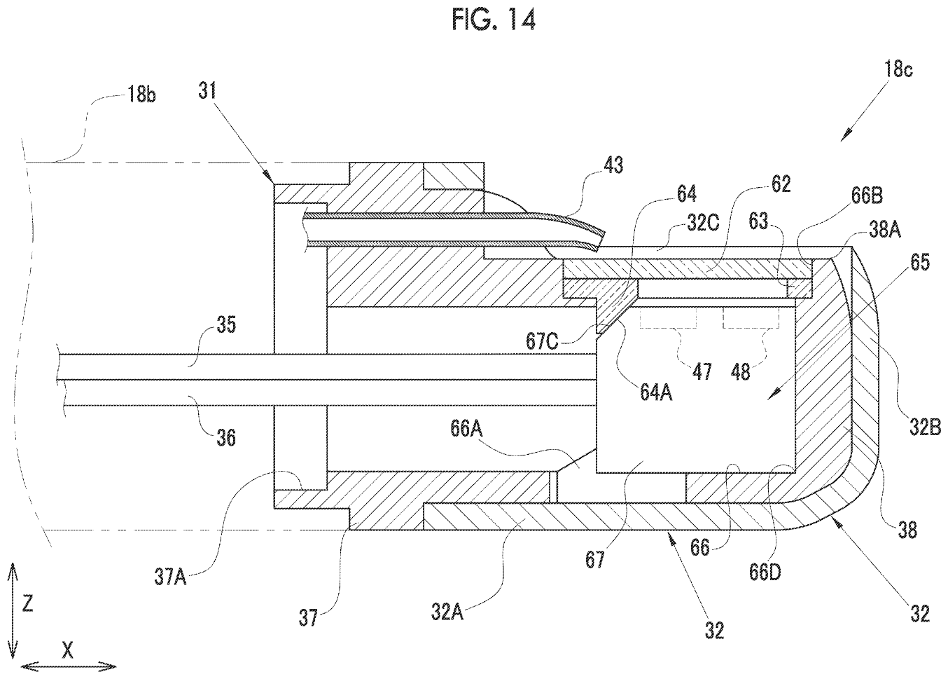

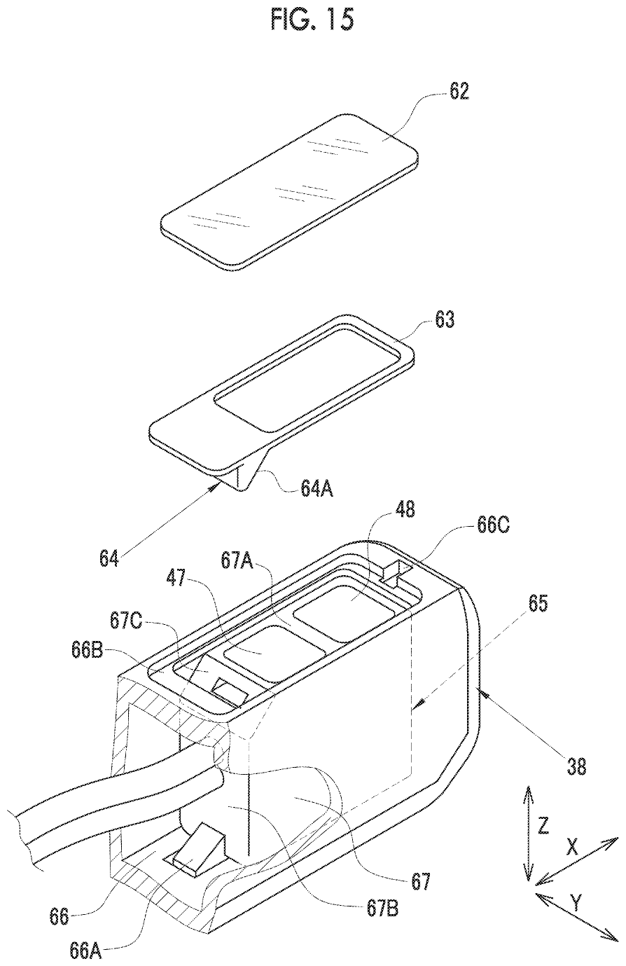

In a cross section orthogonal to the X-axis direction, an inner peripheral surface of the imaging unit housing chamber 66 is formed to match the outer shape of the case member 67. In the present embodiment, in a case where the case member 67 is housed in the imaging unit housing chamber 66, the position of the case member 67 is restricted in the Y-axis direction. Restriction of a position in the X-axis direction and the Z-axis direction with respect to the imaging unit housing chamber 66 will be described later. In the imaging unit housing chamber 66, a locking claw 66A (see FIGS. 14 and 15) is formed. The locking claw 66A locks the proximal end surface of the case member 67. The imaging unit housing chamber 66 communicates with the first opening portion 37A, as with the imaging unit housing chamber 46 of the first embodiment.

In a case where the case member 67 of the imaging unit 65 is to be housed in the imaging unit housing chamber 66, as with the first embodiment, the case member 67 is pushed into the imaging unit housing chamber 66 through the first opening portion 37A such that a distal end surface of the case member 67 is inserted first, for example. The locking claw 66A is elastically deformed by being pressed by the case member 67 and is bent toward the outside of the imaging unit housing chamber 66. Therefore, the locking claw 66A does not hinder the movement of the case member 67. In a case where the case member 67 climbs over the locking claw 66A, the locking claw 66A restores the original shape thereof because of the elastic force thereof and locks the proximal end surface 67B of the case member 67.

As described above, after the case member 67 is housed in the imaging unit housing chamber 66, the packing 63 and the pressing member 64 are interposed between the lens cover 62 and the case member 67 so that the case member 67 is held. The lens cover 62 is fixed to the lens cover opening portion 66B by adhesion or the like using an adhesive. The packing 63 is formed of an elastic material such as rubber or a soft resin, and is a quadrangular frame-shaped elastic body matching an outer periphery of the lens cover 62. The packing 63 is sandwiched between the lens cover 62 and the lens cover opening portion 66B to come into close contact with the lens cover 62 and the lens cover opening portion 66B. Accordingly, the imaging unit 65 is housed in the partition wall portion 38 in an airtight or liquidtight state with respect to the external space.

The pressing member 64 is formed integrally with the packing 63. The pressing member 64 protrudes from a lower surface of the packing 63 and an inclined surface 64A (see FIG. 14) that comes into contact with the chamfer portion 67C of the case member 67 is formed at the pressing member 64.

As shown in FIG. 14, the pressing member 64 is sandwiched between the lens cover 62 and the case member 67 of the imaging unit 65. In a state where the pressing member 64 is sandwiched between the lens cover 62 and the case member 67, the inclined surface 64A is in close contact with the chamfer portion 67C. Accordingly, the chamfer portion 67C of the case member 67 is pressed by the elastic force of the pressing member 64 so that the case member 67 comes into contact with a lower end surface and a distal end surface of the imaging unit housing chamber 66. Therefore, in a state where the lens cover 62 is held at the lens cover opening portion 66B, the position of the case member 67 is restricted in the X-axis direction and the Z-axis direction. In a case where the chamfer portion 67C is on a distal end side of the case member 67, the case member 67 comes into contact with the lower end surface and the proximal end surface of the imaging unit housing chamber 66 because of the elastic force of the pressing member 64. A position where the case member 67 comes into contact with the imaging unit housing portion 66 because of the elastic force of the pressing member 64 is not limited thereto and the case member 67 may come into contact with a corner portion 66D of the imaging unit housing chamber 66 that is positioned diagonally opposite to the chamfer portion 67C.

As described above, in a case where the packing 63 and the pressing member 64 are interposed between the lens cover 62 and the case member 67 and the lens cover 62 is held to be attachable and detachable with respect to the lens cover opening portion 66B, the imaging unit 65 is held to be attachable and detachable with respect to the imaging unit housing chamber 66.

A space between the lens cover 62 and the case member 67 may be filled with a sealing material instead of the packing 63. In this case, the sealing material is preferably formed of transparent rubber or resin. In addition, the packing 63 and the pressing member 64 may be configured separately from each other.

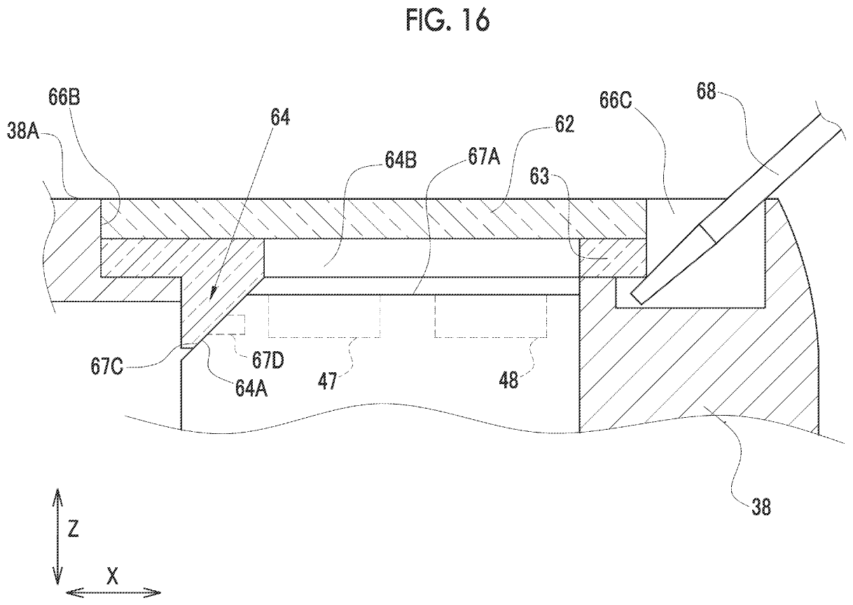

A structure for holding the lens cover 62 to be attachable and detachable with respect to the lens cover opening portion 66B will be described with reference to FIGS. 15 and 16. A jig cutout portion 66C is formed in the vicinity of a distal end portion of the lens cover opening portion 66B in the X-axis direction. The jig cutout portion 66C adjoins the lens cover 62 and is formed up to a position below a lower surface side of the lens cover 62. As a result, even in a case where the lens cover 62 is fixed to the lens cover opening portion 66B by adhesion or the like, as shown in FIG. 16, it is possible to lift up a distal end side of the lens cover 62 by inserting a jig 68, of which a distal end is thin and flat like a flat-bladed screwdriver, into the jig cutout portion 66C. That is, it is possible to remove the lens cover 62 with respect to the lens cover opening portion 66B.

A step of separating the imaging unit 65 from the imaging unit housing chamber 46 after use of the endoscope 12 will be described. First, the lens cover 62 is removed from the lens cover opening portion 66B by means of the jig 68 or the like. In addition, at the time of removal of the lens cover 62 or after removal of the lens cover 62, the packing 63 and the pressing member 64 are removed from the lens cover opening portion 66B and the imaging unit housing chamber 66, respectively. Since the packing 63 and the pressing member 64 are formed of a soft material, the packing 63 and the pressing member 64 can be easily removed.

In a case where the lens cover 62 is removed and the packing 63 and the pressing member 64 are removed, the case member 67 of the imaging unit 65 is exposed through the lens cover opening portion 66B (a state as shown in FIG. 15). Accordingly, in a case where the case member 67 held in the imaging unit housing chamber 66 is pulled toward the lens cover opening portion 66B side, the imaging unit 65 can be separated from the imaging unit housing chamber 66 through the lens cover opening portion 66B.

In addition, in the present embodiment, the jig cutout portion 67D is formed at the chamfer portion 67C of the case member 67. Therefore, it is possible to lift up a proximal end side of the case member 67 by inserting a jig into the jig cutout portion 67D. That is, it is possible to easily separate the imaging unit 65 from the imaging unit housing chamber 66 by pulling the imaging unit 65.

As described above, the imaging unit 65 is housed and attachably and detachably held in the distal end portion body 61 in an airtight or liquidtight state with respect to the external space. Therefore, it is possible to easily remove and reuse the imaging unit 65 after use of the endoscope 12 and components of the endoscope 12 other than the imaging unit 65 can be made disposable. In addition, since the reused imaging unit 65 is housed in the distal end portion body 61 in the airtight or liquidtight state with respect to the external space, infection caused by a virus or the like can be prevented.

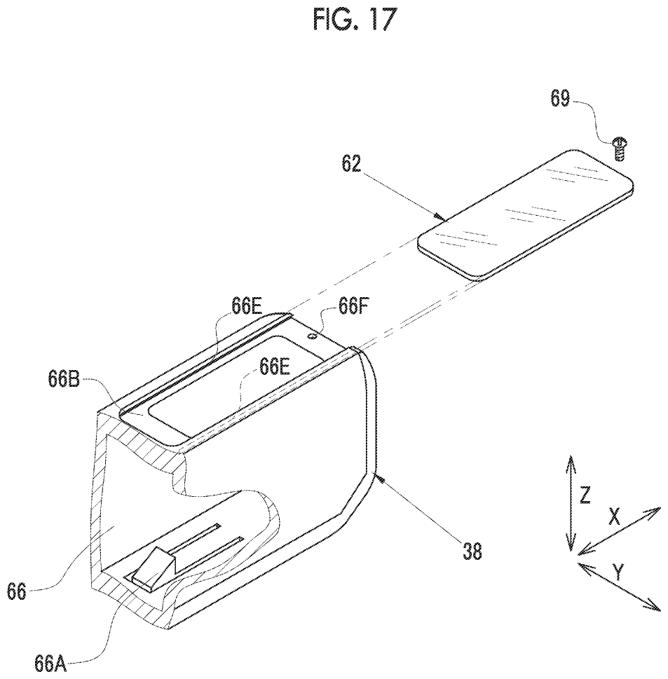

The structure for holding the lens cover 62 to be attachable and detachable with respect to the lens cover opening portion 66B is not limited to that in the second embodiment and a structure in which a retaining member is used as shown in FIG. 17 may also be adopted. In this case, the lens cover opening portion 66B continues to a distal end side of the partition wall portion 38. In addition, a pair of rail grooves 66E (rail portions) is formed at the lens cover opening portion 66B such that the rail grooves are formed at both of right and left end portions in the Y-axis direction. The rail grooves 66E are disposed along the X-axis direction. The lens cover 62 can be made slidable from the distal end side of the partition wall portion 38 along the X-axis direction with both side edges of the lens cover 62 fitted to the rail grooves 66E and the position of the lens cover 62 can be restricted in the Y-axis direction and the Z-axis direction.

In a case where both side edges of the lens cover 62 are fitted to the rail grooves 66E, the lens cover 62 is disposed at a position facing the imaging unit 65. A configuration in which the lens cover 62 is slidable along the X-axis direction is not limited to the configuration as described above and a configuration in which both side edges of the lens cover 62 are provided with recess grooves extending in the X-axis direction and both of right and left end portions of the lens cover opening portion 66B are provided with protruding portions (rail portions) fitted to the recess grooves may also be adopted.

A screw hole 66F is formed in the vicinity of a distal end of the lens cover opening portion 66B. After the lens cover 62 of which both side edges are fitted to the rail grooves 66E is slid to a proximal end of the lens cover opening portion 66B, a screw 69 as a retaining member is screwed into the screw hole 66F.

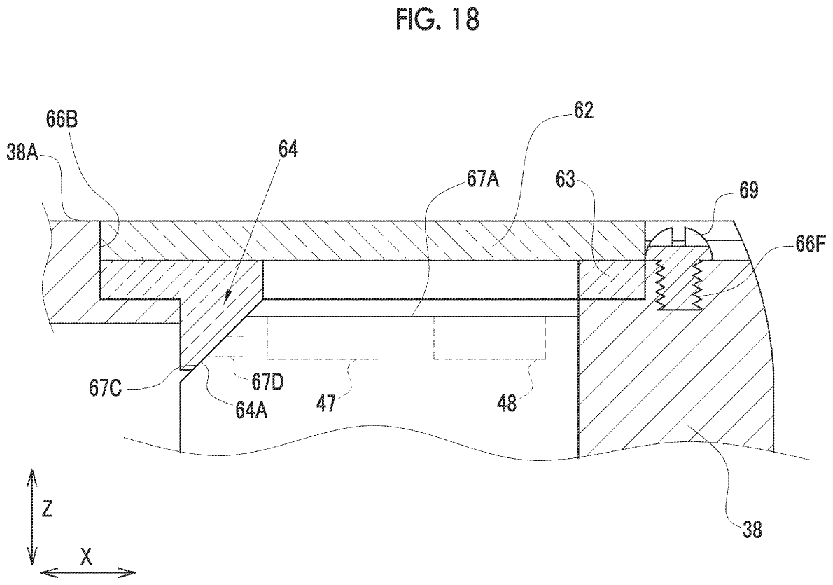

As shown in FIG. 18, the screw 69 screwed into the screw hole 66F is disposed at a position where the screw 69 comes into contact with the distal end of the lens cover 62. Accordingly, the position of the lens cover 62 can be restricted in a slide direction, that is, the X-axis direction. As described above, in a case where the screw 69 is screwed into the screw hole 66F, the lens cover 62 can be held at the lens cover opening portion 66B and in a case where the screw 69 is removed from the screw hole 66F, the lens cover 62 can be separated from the lens cover opening portion 66B.

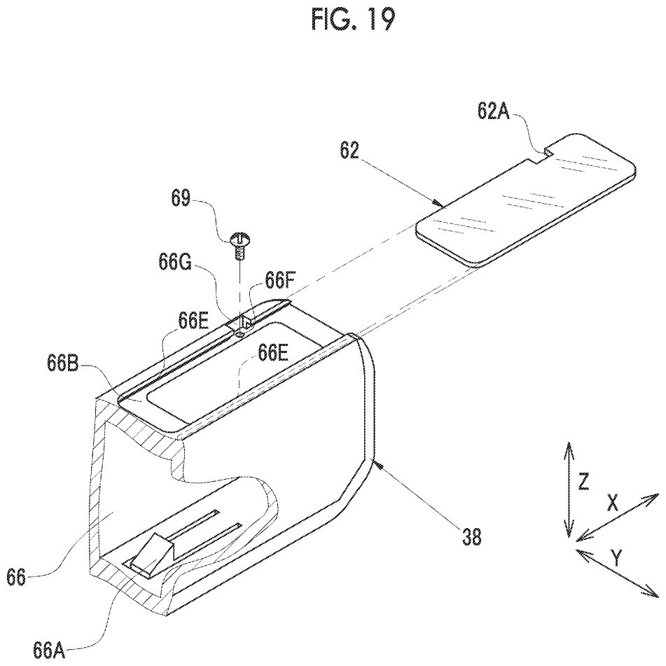

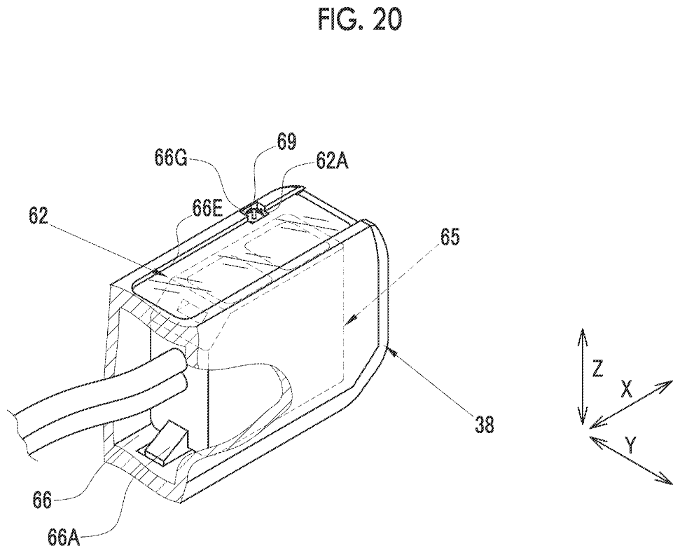

The screw 69 as the retaining member may not be disposed at a position where the screw 69 comes into contact with the distal end of the lens cover 62 as long as the screw 69 is disposed on the trajectory of the lens cover 62 sliding with respect to the rail grooves 66E. In a modification example shown in FIG. 19 and FIG. 20, a cutout portion 66G obtained by cutting out an intermediate portion of the rail groove 66E is formed and the screw hole 66F is disposed in the cutout portion 66G. In addition, a cutout portion 62A to be aligned with the cutout portion 66G is formed in the lens cover 62 as well. After the lens cover 62 is slid to a position where the cutout portion 62A and the cutout portion 66G coincide with each other as shown in FIG. 20, the screw 69 is screwed into the screw hole 66F. Accordingly, the position of the lens cover 62 can be restricted in the slide direction, that is, the X-axis direction.

The retaining member in the modification example of the second embodiment is not limited to the screw 69 and may be a fitting pin to be fitted to a fitting recess portion formed at the lens cover opening portion 66B as long as the position of the lens cover 62 can be restricted in the slide direction.

### Third Embodiment

In the first and second embodiments, a configuration in which the imaging units 45 and 65 are attachable and detachable with respect to the distal end portion bodies 31 and 61 has been used as an example. However, the present invention is not limited thereto and in a third embodiment which will be described below, an imaging unit is separated together with a distal end portion body. The same components and members as those of the distal end portion 18c of the first and second embodiments are denoted by the same reference numerals and the descriptions thereof will be omitted.

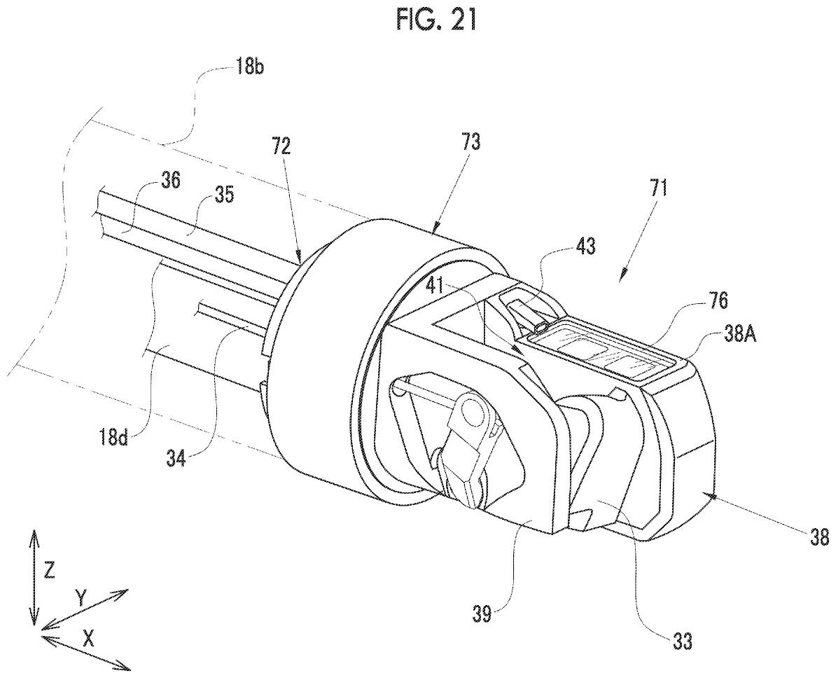

As shown in FIG. 21, a distal end portion body 71 is connected to the bendable portion 18b via a fixing member 72 and a ring member 73. The fixing member 72 is coupled to the distal end side of the bendable portion 18b as with the disk portion 37 of the first and second embodiments. The fixing member 72 is fixed to the bendable piece positioned on the most distal end side among the plurality of bendable pieces forming the bendable portion 18b by, for example, screwing or adhesion or the like using an adhesive.

The fixing member 72 includes a male screw portion 72A that is formed in a substantially annular shape and is formed on an outer peripheral surface and a pair of slits 72B that is cut out over an area from a distal end surface to a proximal end surface in the X-axis direction. The slits 72B engage with engaging claws 74B which will be described below.

The distal end portion body 71 includes a disk portion 74 and the pair of partition wall portions 38 and 39. The pair of partition wall portions 38 and 39 is provided to protrude from the disk portion 74 in the X-axis direction. The distal end portion body 71 is made of, for example, an elastic resin material. In addition, the above-described elevator housing portion 41 housing the elevator 33 is provided between the partition wall portion 38 and the partition wall portion 39.

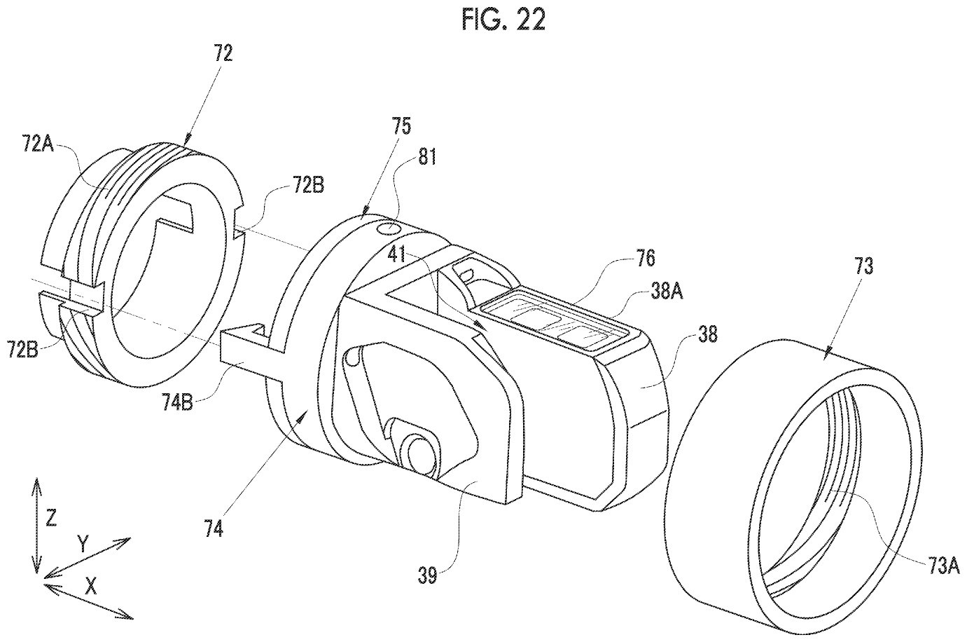

As shown in FIG. 22, at the disk portion 74, an opening portion 74A (see FIGS. 21 and 22) and a pair of engaging claws 74B are formed. The engaging claws 74B are disposed at both of right and left end portions of the disk portion 74 in the Y-axis direction. The engaging claws 74B are inserted into the slits 72B and engage with the slits 72B such that the engaging claws 74B sandwich the slits 72B from both sides by means of the elastic force thereof.

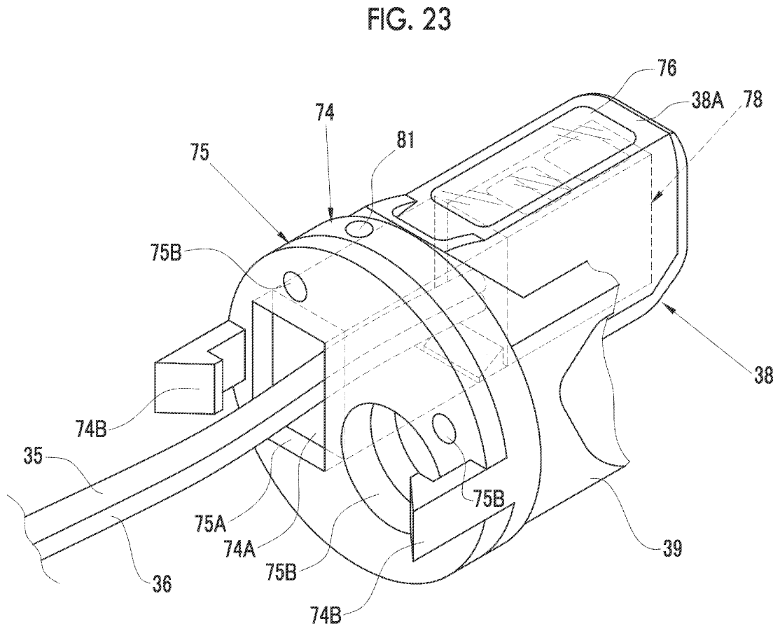

As shown in FIG. 23, packing 75 is provided on a proximal end side of the disk portion 74. The packing 75 is formed of rubber or a soft resin, and is formed in a circular shape having the same outer diameter as the disk portion 74. The packing 75 is sandwiched between the disk portion 74 and the fixing member 72 to come into close contact with the fixing member 72 and the disk portion 74. Accordingly, it is possible to maintain an airtight or liquidtight state between the distal end portion body 71 and the fixing member 72. In addition, the packing 75 includes an opening portion 75A that communicates with the opening portion 74A of the disk portion 74 and a through-hole 75B for insertion of an operation wire, an air and water supply channel, a treatment tool channel, and the like.

The ring member 73 is formed in an annular shape having an outer diameter larger than that of the fixing member 72, and includes a female screw portion 73A provided on an inner peripheral surface. After the slits 72B and the engaging claws 74B engage with each other so that the distal end portion body 71 and the fixing member 72 are coupled to each other, the female screw portion 73A of the ring member 73 is screwed onto the male screw portion 72A of the fixing member 72. The ring member 73 with the female screw portion 73A screwed onto the male screw portion 72A is in contact with the engaging claws 74B. Accordingly, the movement of the engaging claws 74B is hindered, so that engagement between the slits 72B and the engaging claws 74B is maintained. That is, a state where the distal end portion body 71 and the fixing member 72 are connected to each other is maintained and a state where the packing 75 keeps the inside of the distal end portion body 71 airtight or liquidtight is maintained.

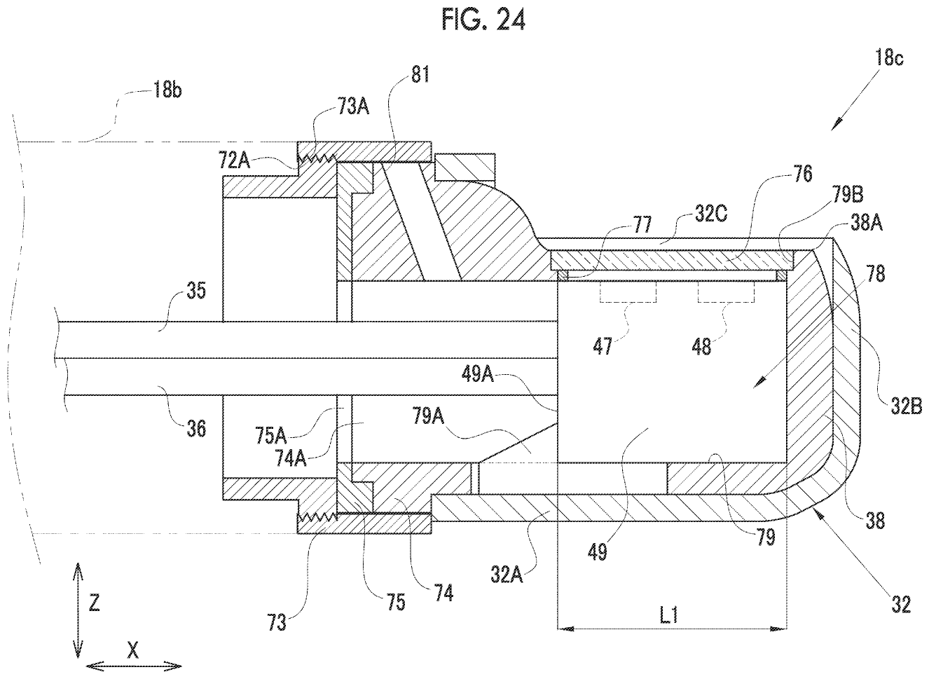

As shown in FIG. 24, the partition wall portion 38 includes a lens cover 76, the air and water supply nozzle 43, packing 77 (see FIG. 6), and an imaging unit 78. An imaging unit housing chamber 79 (a holding portion) is provided in the partition wall portion 38. The imaging unit housing chamber 79 is formed in a substantially quadrangular tubular shape of which a distal end side is sealed.

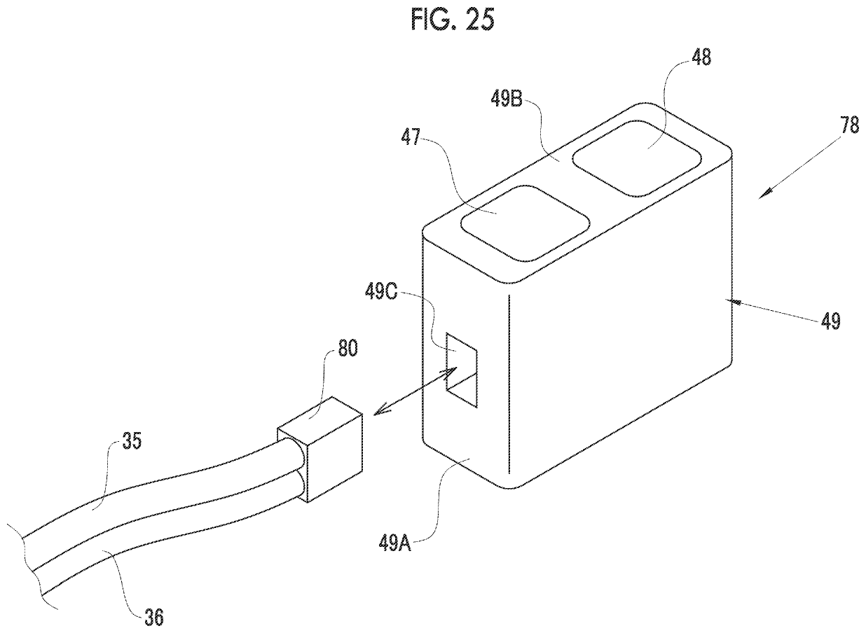

The imaging unit 78 is configured such that it is possible to pull the imaging unit 78 to the proximal end side in the X-axis direction and to separate the imaging unit 78 from the imaging unit housing chamber 79. The imaging unit 78 is composed of the image sensor 47, the light source 48, the signal cable 35, the electric cable 36, the case member 49, and a relay connector 80 (see FIG. 25).

As shown in FIG. 25, the case member 49 is formed in a substantially quadrangular columnar shape. The image sensor 47 and the light source 48 are exposed through an upper end surface 49B of the case member 49 and are provided at positions where the image sensor 47 and the light source 48 face the lens cover 76 in a case where the case member 49 is housed in the imaging unit housing chamber 79. Accordingly, at least a portion of the imaging unit 78 is exposed through the lens cover 76.

The relay connector 80 is provided to be attachable and detachable with respect to the case member 49. A fitting hole 49C to which the relay connector 80 is fitted is formed in the proximal end surface 49A of the case member 49. The signal cable 35 and the electric cable 36 are connected to the case member 49 via the relay connector 80. In a case where the relay connector 80 and the fitting hole 49C of the case member 49 are fitted to each other, the image sensor 47 is electrically connected to the signal cable 35 and the light source 48 is electrically connected to the electric cable 36. Accordingly, the imaging unit 78 can acquire an imaging signal by means of the image sensor 47 and irradiate an observation target with illumination light by means of the light source 48.

In a case where the relay connector 80 is connected to the case member 49, the relay connector 80 is disposed inside the imaging unit housing chamber 79 together with the case member 49 and as with the above-described first embodiment, the light source 48 provided in the imaging unit 78 is preferably composed of a light emitting element such as a laser diode (LD) or a light emitting diode (LED).

An inner peripheral surface of the imaging unit housing chamber 79 is formed to match the outer shape of the case member 49. Accordingly, in a case where the case member 49 is housed in the imaging unit housing chamber 79, the position of the case member 49 is restricted in the Z-axis direction and the Y-axis direction. The imaging unit housing chamber 79 communicates with the opening portion 74A of the disk portion 74 and the opening portion 75A of the packing 75. Accordingly, in a case where the case member 49 is to be housed in the imaging unit housing chamber 79 from the external space and in a case where the case member 49 is to be separated from the imaging unit housing chamber 79 and moved to the external space, the case member 49 can be moved through the first opening portion 74A and the opening portion 75A.

In the imaging unit housing chamber 79, a locking claw 79A as a locking portion is formed. The locking claw 79A is disposed to be separated from a distal end of the imaging unit housing chamber 79 by an interval corresponding to the long side L1 (the length in the X-axis direction) of the case member 49. Since the case member 49 is locked by the locking claw 79A, the position of the case member 49 is restricted in the X-axis direction in a case where the case member 49 is housed in the imaging unit housing chamber 79. As described above, the case member 49 of which the position is restricted by the inner peripheral surface of the imaging unit housing chamber 79 and the locking claw 79A is attachably and detachably held in the imaging unit housing chamber 79. A locking portion locking the imaging unit 78 is not limited to the locking claw 79A as described above as long as the movement of the imaging unit 78 is restricted by the locking portion. For example, an opening portion may be formed in the imaging unit housing chamber 79 and the imaging unit 78 may be locked by a locking pin fitted to the opening portion.

A lens cover opening portion 79B is formed at an upper portion of the imaging unit housing chamber 79. The lens cover opening portion 79B is an opening portion that is cut out from the upper end surface 38A of the partition wall portion 38 in the Z-axis direction to an upper surface of the imaging unit housing chamber 79 and of which the cross section is quadrangular.

The lens cover 76 is formed of transparent glass or a resin material and is formed in a rectangular plate-like shape. The lens cover 76 is fitted to the lens cover opening portion 79B and is fixed to the partition wall portion 38 by adhesion using an adhesive or the like. Therefore, the imaging unit housing chamber 79 houses the case member 49 of the imaging unit 78 with the case member 49 being at a position facing the lens cover 76. The image sensor 47 provided in the imaging unit 78 can observe, via the lens cover 76, an upper visual field region in the Z-axis direction at which the opening window 32C is open.

In addition, the packing 77 is sandwiched between the lens cover 76 and the case member 49. The packing 77 is an elastic member that is formed of rubber or a soft resin, is slightly smaller than the lens cover 76, and has a quadrangular frame-like shape matching the inner peripheral surface of the imaging unit housing chamber 79. Therefore, in a state where the packing 77 is sandwiched between the lens cover 76 and the case member 49, the packing 77 comes into close contact with the upper end surface of the case member 49 and the inner peripheral surface of the imaging unit housing chamber 46. Because of the packing 75 and the packing 77, the imaging unit 78 is housed in the partition wall portion 38 in an airtight or liquidtight state with respect to the external space.

A space between the lens cover 76 and the case member 49 may be filled with a sealing material instead of the packing 77. In this case, the sealing material is preferably formed of transparent rubber or resin.

The distal end portion body 71 includes an unlocking hole 81 for unlocking the case member 49 locked by the locking claw 79A. Specifically, the unlocking hole 81 is disposed above the imaging unit housing chamber 79 in the Z-axis direction and is disposed at a position on the proximal end side of the imaging unit housing chamber 79 in the X-axis direction. The unlocking hole 81 is disposed in an outer peripheral surface of the disk portion 74 onto which the ring member 73 is mounted (refer to FIGS. 22 and 23) and the unlocking hole 81 communicates with the imaging unit housing chamber 79. In a case where the case member 49 locked by the locking claw 79A is to be unlocked, the case member 49 is unlocked after the ring member 73 is removed. Accordingly, the imaging unit housing chamber 79 can be maintained in an airtight or liquidtight state in a case where the ring member 73 is in a mounted state, and the airtight or liquidtight state ends in a case where the ring member 73 is removed.

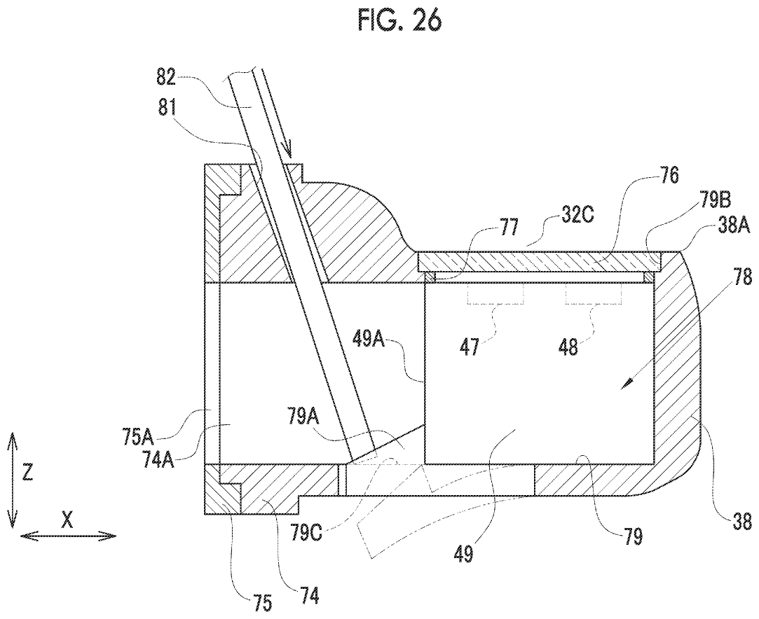

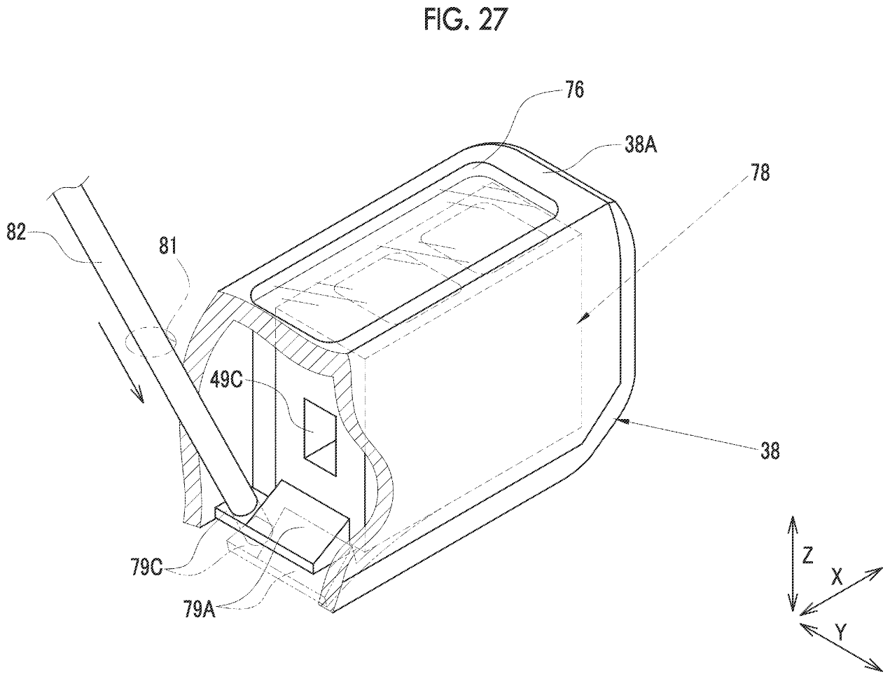

As shown in FIG. 27, a pressing target piece 79C is integrally formed with the locking claw 79A. The pressing target piece 79C is disposed such that a step is provided between the pressing target piece 79C and the locking claw 79A. It is preferable that at least a portion of the pressing target piece 79C is positioned at an end in a direction in which the unlocking hole 81 penetrates. Accordingly, a long and narrow columnar jig 82 (see FIGS. 26 and 27) inserted into the distal end portion body 71 can easily press the pressing target piece 79C through the unlocking hole 81. That is, the locking claw 79A integrated with the pressing target piece 79C can be displaced. In addition, the unlocking hole 81 and the pressing target piece 79C are disposed to be offset from the imaging unit 78 in the Y-axis direction.

A step of separating the distal end portion body 71 from the fixing member 72 after use of the endoscope 12 and a step of separating the imaging unit 78 from the imaging unit housing chamber 79 will be described. First, in the step of separating the distal end portion body 71 from the fixing member 72, the male screw portion 72A of the fixing member 72 and the female screw portion 73A of the ring member 73 are unscrewed from each other. Then, the unscrewed ring member 73 is removed from the distal end portion body 71 and the fixing member 72.

In addition, the relay connector 80 is separated from the case member 49 of the imaging unit 78 before the engaging claws 74B and the slits 72B are disengaged from each other. That is, the signal cable 35, the electric cable 36, and the case member 49 are disconnected from each other. The signal cable 35 and the electric cable 36 are connected to connectors (not shown) through the insertion part 18 and the operation part 19 and the connectors need to be removed from the signal cable 35 and the electric cable 36 in a case where there is no relay connector 80. However, in the present embodiment, since the relay connector 80 is provided, the insertion part 18, the operation part 19, the connectors (not shown), and the like do not interfere with each other in a case where the engaging claws 74B and the slits 72B are disengaged from each other.

In a case where the ring member 73 is removed from the distal end portion body 71 and the fixing member 72, the engaging claws 74B are exposed. In addition, in a case where the engaging claws 74B are opened to be disengaged from the slits 72B, the fixing member 72 and the distal end portion body 71 are decoupled from each other and thus the distal end portion body 71 can be separated from the fixing member 72. After the distal end portion body 71 is separated from the fixing member 72, the step of separating the imaging unit 78 from the imaging unit housing chamber 79 is performed.

As shown in FIGS. 26 and 27, the jig 82 is inserted into the distal end portion body 71 through the unlocking hole 81 to press the pressing target piece 79C. In this case, the locking claw 79A pressed together with the pressing target piece 79C is elastically deformed and is bent toward the outside of the imaging unit housing chamber 79 (a position shown by a two-dot chain line). Therefore, the locking claw 79A allows the movement of the case member 49. Accordingly, it is possible to pull the imaging unit 78 to the proximal end side in the X-axis direction and to separate the imaging unit 78 from the imaging unit housing chamber 79. In addition, since the unlocking hole 81 and the pressing target piece 79C are disposed to be offset from the imaging unit 78 in the Y-axis direction, the jig 82 pressing the pressing target piece 79C does not hinder the movement of the imaging unit 78.

As described above, the imaging unit 78 is housed in the distal end portion body 71 in an airtight or liquidtight state with respect to the external space and the distal end portion body 71 is held to be attachable and detachable with respect to the fixing member 72. Therefore, it is possible to easily remove and reuse the imaging unit 78 after use of the endoscope 12 and components of the endoscope 12 other than the imaging unit 78 can be made disposable. In addition, since the reused imaging unit 78 is housed in the distal end portion body 71 in the airtight or liquidtight state with respect to the external space, infection caused by a virus or the like can be prevented.

In addition, since the imaging unit 78 is separated from the fixing member 72 integrally with the imaging unit housing chamber 79, the imaging unit 78 can be easily removed from the bendable portion 18b. Furthermore, since the distal end portion body 71 is provided with the unlocking hole 81, the imaging unit 78 can be easily separated from the imaging unit housing chamber 79.

In the third embodiment, an example in which the jig 82 is used to press the locking claw 79A and to unlock the imaging unit 78 has been described. However, the present invention is not limited thereto and, for example, the jig 82 may be used to press and damage the locking claw 79A and to remove at least a portion of the locking claw 79A. Accordingly, the imaging unit 78 is unlocked as in the third embodiment. In addition, in the third embodiment, the distal end portion body 71 is configured to attachably and detachably engage with the fixing member 72 fixed to the bendable portion 18b. However, the present invention is not limited thereto and the distal end portion body 71 may be configured to attachably and detachably engage with the bendable portion 18b. In this case, it is preferable that a bendable piece that forms the bendable portion 18b and is positioned on the most distal end side engages with the distal end portion body 71.

### Fourth Embodiment

In the third embodiment, the imaging unit 78 is separated together with the distal end portion body 71. However, the present invention is not limited thereto and in a fourth embodiment which will be described below, an imaging unit is separated together with a portion of a distal end portion body. The same components and members as those of the distal end portion 18c of the first to third embodiments are denoted by the same reference numerals and the descriptions thereof will be omitted.

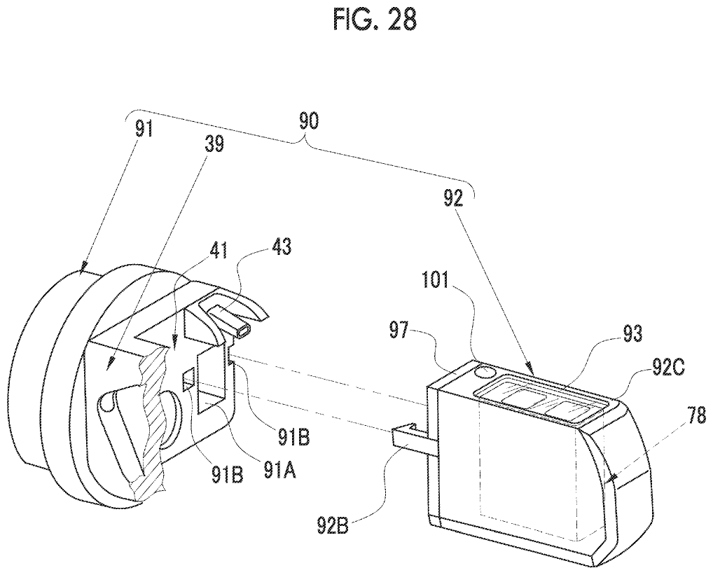

As shown in FIG. 28, a distal end portion body 90 includes a disk portion 91, a partition wall portion 92, and the partition wall portion 39. The partition wall portion 92 and the partition wall portion 39 that form a pair are provided to protrude from the disk portion 91 in the X-axis direction. The disk portion 91 is coupled to the distal end side of the bendable portion 18b as with the disk portion 37 of the first and second embodiments. The disk portion 91 is fixed to the bendable piece positioned on the most distal end side among the plurality of bendable pieces forming the bendable portion 18b by, for example, screwing or adhesion using an adhesive.

The partition wall portion 92 is the same as the partition wall portion 38 of the first and second embodiments except that the partition wall portion 92 is attachable and detachable with respect to the disk portion 91 and the partition wall portion 92 and the partition wall portion 39 are disposed opposite each other in the Y-axis direction. In addition, the elevator housing portion 41 housing the elevator 33 is provided between the partition wall portion 92 and the partition wall portion 39.

The disk portion 91 includes an opening portion 91A and a pair of slits 91B that is cut out over an area from a distal end surface to a proximal end surface in the X-axis direction. The signal cable 35 and the electric cable 36 pass through the opening portion 91A. The slits 91B engage with engaging claws 92B which will be described below.

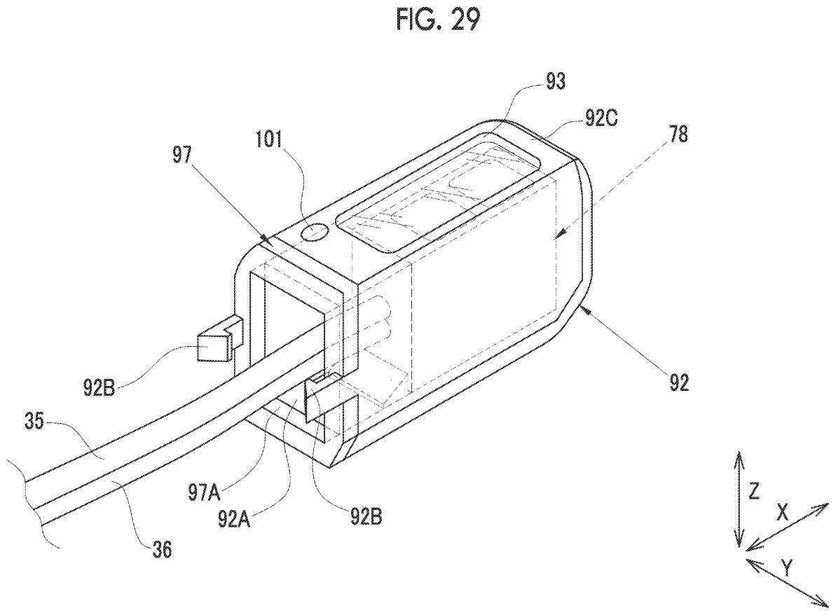

As shown in FIG. 29, the partition wall portion 92 includes a lens cover 93, packing 94 (see FIG. 27), and the imaging unit 78. An imaging unit housing chamber 96 (a holding portion) is provided in the partition wall portion 92. The imaging unit housing chamber 96 is formed in a substantially quadrangular tubular shape of which a distal end side is sealed.

At the partition wall portion 92, an opening portion 92A (see FIGS. 26 and 27) and a pair of engaging claws 92B are formed. The engaging claws 92B are disposed at both of right and left end portions of the partition wall portion 92 in the Y-axis direction. The engaging claws 92B are inserted into the slits 91B and engage with the slits 91B such that the engaging claws 92B sandwich the slits 91B from both sides by means of the elastic force thereof.

Packing 97 is provided on a proximal end side of the partition wall portion 92. The packing 97 is formed of rubber or a soft resin, and is formed in a quadrangular shape having the same outer shape as a proximal end surface of the partition wall portion 92. The packing 97 is sandwiched between the disk portion 91 and the partition wall portion 92 to come into close contact with the disk portion 91 and the partition wall portion 92. Accordingly, the inside of the distal end portion body 90 can be brought into an airtight or liquidtight state. In addition, in the packing 97, an opening portion 97A that communicates with the partition wall portion 92 and the opening portion 92A is formed.

An inner peripheral surface of the imaging unit housing chamber 96 is formed to match the outer shape of the case member 49. Accordingly, in a case where the case member 49 is housed in the imaging unit housing chamber 96, the position of the case member 49 is restricted in the Z-axis direction and the Y-axis direction. The imaging unit housing chamber 96 communicates with the opening portion 92A and the opening portion 97A of the packing 97. Accordingly, in a case where the case member 49 is to be housed in the imaging unit housing chamber 96 from the external space and in a case where the case member 49 is to be separated from the imaging unit housing chamber 96 and moved to the external space, the case member 49 can be moved through the opening portion 92A and the opening portion 97A.

In the imaging unit housing chamber 96, a locking claw 96A is formed. The locking claw 96A is disposed to be separated from a distal end of the imaging unit housing chamber 96 by an interval corresponding to the long side L1 (the length in the X-axis direction) of the case member 49. Since the case member 49 is locked by the locking claw 96A, the position of the case member 49 is restricted in the X-axis direction in a case where the case member 49 is housed in the imaging unit housing chamber 96. As described above, the case member 49 of which the position is restricted by the inner peripheral surface of the imaging unit housing chamber 96 and the locking claw 96A is attachably and detachably held in the imaging unit housing chamber 79.

A lens cover opening portion 96B is formed at an upper portion of the imaging unit housing chamber 96. The lens cover opening portion 96B is an opening portion that is cut out from an upper end surface 92C of the partition wall portion 92 in the Z-axis direction to an upper surface of the imaging unit housing chamber 96 and of which the cross section is quadrangular.

The lens cover 93 is formed of transparent glass or a resin material and is formed in a rectangular plate-like shape. The lens cover 93 is fitted to the lens cover opening portion 96B and is fixed to the partition wall portion 92 by adhesion using an adhesive or the like. Therefore, the imaging unit housing chamber 96 houses the case member 49 of the imaging unit 78 with the case member 49 being at a position facing the lens cover 93. Accordingly, at least a portion of the imaging unit 78 is exposed through the lens cover 93. The image sensor 47 provided in the imaging unit 78 can observe, via the lens cover 93, an upper visual field region in the Z-axis direction at which the opening window 32C is open.

In addition, the packing 94 is sandwiched between the lens cover 93 and the case member 49. The packing 94 is an elastic member that is formed of rubber or a soft resin, is slightly smaller than the lens cover 93, and has a quadrangular frame-like shape matching the inner peripheral surface of the imaging unit housing chamber 96. Therefore, in a state where the packing 94 is sandwiched between the lens cover 93 and the case member 49, the packing 94 comes into close contact with an upper end surface 49B of the case member 49 and the inner peripheral surface of the imaging unit housing chamber 96. Because of the packing 94 and the packing 97, the imaging unit 78 is housed in the partition wall portion 92 in an airtight or liquidtight state with respect to the external space.

A space between the lens cover 93 and the case member 49 may be filled with a sealing material instead of the packing 97. In this case, the sealing material is preferably formed of transparent rubber or resin.

The partition wall portion 92 includes an unlocking hole 101 for unlocking the case member 49 locked by the locking claw 96A. Specifically, the unlocking hole 101 is disposed at the upper end surface 92C of the partition wall portion 92 and is disposed at a position on the proximal end side of the imaging unit housing chamber 96 in the X-axis direction.

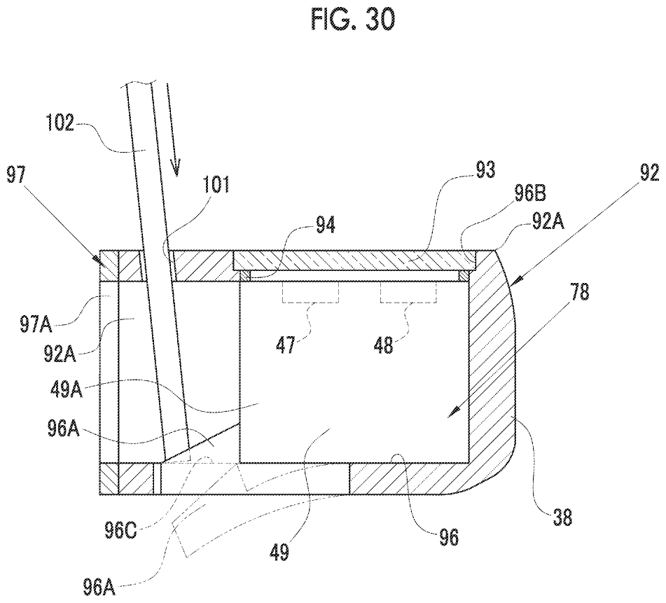

A pressing target piece 96C (see FIG. 30) is integrally formed with the locking claw 96A. The pressing target piece 96C has the same configuration as the pressing target piece 79C in the third embodiment and is disposed such that a step is provided between the pressing target piece 96C and the locking claw 96A. It is preferable that at least a portion of the pressing target piece 96C is positioned at an end in a direction in which the unlocking hole 101 penetrates. Accordingly, a long and narrow columnar jig 102 (see FIG. 30) inserted into the partition wall portion 92 can easily press the pressing target piece 96C through the unlocking hole 101. That is, the locking claw 96A integrated with the pressing target piece 96C can be displaced. In addition, the unlocking hole 101 and the pressing target piece 96C are disposed to be offset from the imaging unit 78 in the Y-axis direction.

A step of separating the partition wall portion 92 from the disk portion 91 after use of the endoscope 12 and a step of separating the imaging unit 78 from the imaging unit housing chamber 96 will be described. First, the relay connector 80 is separated from the case member 49 of the imaging unit 78 before the engaging claws 92B and the slits 91B are disengaged from each other as in the third embodiment. Accordingly, the insertion part 18, the operation part 19, the connectors (not shown), and the like do not interfere with each other in a case where the engaging claws 92B and the slits 91B are disengaged from each other.

After the relay connector 80 is separated from the case member 49, for example, a jig or the like having a thin distal end is used to press the engaging claws 92B from a proximal end side of the slits 91B. In addition, in a case where the engaging claws 92B are opened to be disengaged from the slits 91B, the disk portion 91 and the partition wall portion 92 are decoupled from each other and thus the partition wall portion 92 can be separated from the disk portion 91. After the partition wall portion 92 is separated from the disk portion 91, the step of separating the imaging unit 78 from the imaging unit housing chamber 96 is performed.

As shown in FIG. 30, the jig 102 is inserted into the partition wall portion 92 through the unlocking hole 101 to press the pressing target piece 96C. In this case, the locking claw 96A pressed together with the pressing target piece 96C is elastically deformed and is bent toward the outside of the imaging unit housing chamber 96 (a position shown by a two-dot chain line). Therefore, the locking claw 96A allows the movement of the case member 49. Accordingly, it is possible to pull the imaging unit 78 to the proximal end side in the X-axis direction and to separate the imaging unit 78 from the imaging unit housing chamber 96. In addition, since the unlocking hole 101 and the pressing target piece 96C are disposed to be offset from the imaging unit 78 in the Y-axis direction, the jig 102 pressing the pressing target piece 96C does not hinder the movement of the imaging unit 78.

As described above, the imaging unit 78 is housed in the partition wall portion 92, which is a portion of the distal end portion body 90, in an airtight or liquidtight state with respect to the external space and the partition wall portion 92 is held to be attachable and detachable with respect to the disk portion 91. Therefore, it is possible to easily remove and reuse the imaging unit 78 after use of the endoscope 12 and components of the endoscope 12 other than the imaging unit 78 can be made disposable. In addition, since the reused imaging unit 78 is housed in the airtight or liquidtight state with respect to the external space, infection caused by a virus or the like can be prevented.

In addition, since the imaging unit 78 is separated from the fixing member 72 integrally with the imaging unit housing chamber 96, the imaging unit 78 can be easily removed from the bendable portion 18b. Furthermore, since the partition wall portion 92, which is a portion of the distal end portion body 90, is provided with the unlocking hole 101, the imaging unit 78 can be easily separated from the imaging unit housing chamber 96.

In the fourth embodiment, an example in which the jig 102 is used to press the locking claw 96A and to unlock the imaging unit 78 has been described. However, the present invention is not limited thereto and, for example, the jig 102 may be used to press and damage the locking claw 96A and to remove at least a portion of the locking claw 96A. Accordingly, the imaging unit 78 is unlocked as in the fourth embodiment. In addition, in the third and fourth embodiments, the jigs 82 and 102 inserted through the unlocking holes 81 and 101 press the locking claws 79A and 96a so that the imaging unit 78 locked by the locking claws 79A and 96A is unlocked. However, as with the first embodiment, the jig 50 that pulls the imaging unit 78 may be used to unlock the imaging unit 78 locked by the locking claws 79A and 96A and to separate the imaging unit 78 from the imaging unit housing chambers 79 and 96. In this case, it is preferable that the imaging unit 78 is provided with the connection opening portion 52 as with the imaging unit 45 of the first embodiment.

In addition, in the third and fourth embodiments, the imaging unit 78 can be separated from the imaging unit housing chambers 79 and 96 by being pulled toward the proximal end side in the X-axis direction. However, the present invention is not limited thereto and, as with the second embodiment, the lens covers 76 and 93 may be attachable and detachable with respect to the lens cover opening portions 79B and 96B so that the imaging unit 78 can be separated from the imaging unit housing chamber 79 through the lens cover opening portion 79B. In addition, as a configuration in which the lens covers 76 and 93 are attachable and detachable with respect to the lens cover opening portions 79B and 96B, a configuration in which the lens cover opening portions 79B and 96B are provided with the jig cutout portion 66C as with the second embodiment and a configuration in which the lens covers 76 and 93 are slidable with respect to the lens cover opening portions 79B and 96B and the positions of the lens covers 76 and 93 are restricted by the retaining member are preferable.

In addition, in the first and second embodiments, a configuration in which the imaging units 45 and 65 are separated from the distal end portion body in a state where a signal cable and a electric cable are connected to the imaging units 45 and 65 has been described. However, the present invention is not limited thereto and, as with the third and fourth embodiments, the imaging units 45 and 65 may be provided with a relay connector the signal cable and the electric cable may be disconnected before the imaging units 45 and 65 are separated from the distal end portion body.

In each of the above-described embodiments, the imaging unit includes the electric cable 36 and the light source 48. However, the present invention is not limited thereto and the electric cable 36 and the light source 48 may be omitted from the imaging unit in each of the above-described embodiments (only the signal cable 35 and the image sensor 47 may be provided as electric components).

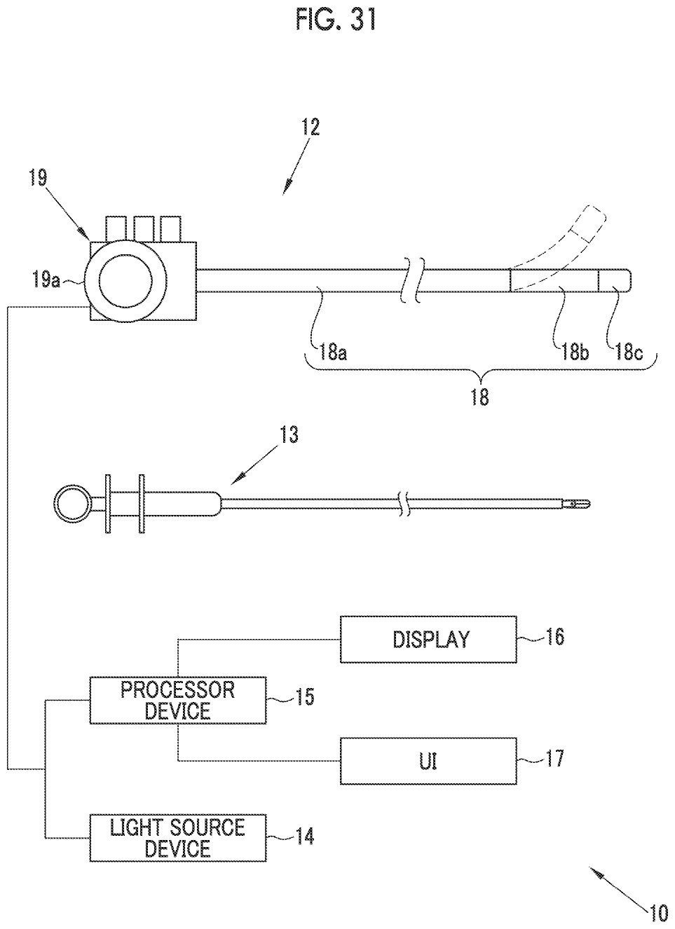

In addition, the configuration of the imaging unit is not limited to the above-described configuration. A light guide may be provided instead of the electric cable 36 and the case members 49 and 67 may be provided with an illumination optical system instead of the light source 48. Furthermore, in the case of such a modification example, it is preferable that the endoscope system 10 includes a light source device 14 in addition to the configuration as in each of the above-described embodiments as shown in FIG. 31. The light source device 14 emits illumination light with which the observation target is irradiated. The endoscope 12 is optically connected to the light source device 14 and is electrically connected to the processor device 15.

In addition, in the case of such a modification example, it is preferable that the imaging unit is provided with no relay connector 80 and the imaging unit is electrically connected to the image sensor 47 and the signal cable 35 and is optically connected to the illumination optical system and the light guide. The signal cable 35 and the light guide protrude from the proximal end surface of the case members 49 and 67 and are respectively connected to the processor device 15 and the light source device 14 through the insertion part 18, the operation part 19, a connector (not shown), and the like. The processor device 15 performs image processing or the like on the imaging signal acquired by the image sensor 47 and causes the display 16 to display the observation image. The light guide is formed of an optical fiber cable or the like, transmits the illumination light emitted from the light source device 14, and irradiates the observation target with the illumination light through the illumination optical system.

In addition, in each of the above-described embodiments, the imaging unit includes the illumination optical system or the light source. However, the imaging unit does not need to be provided with the illumination optical system or the light source. In this case, the illumination optical system or the light source may be provided at the distal end portion of the endoscope as a component separate from the imaging unit.

In addition, in each of the above-described embodiments, the present invention is applied to a side-viewing endoscope. However, the present invention is not limited thereto and the present invention may be applied to a direct-viewing type endoscope.

Explanation of References
10: endoscope system
12: endoscope
13: treatment tool
14: light source device
15: processor device
16: display
17: user interface (UI)
18: insertion part
18a: soft portion
18b: bendable portion
18c: distal end portion
18d: treatment tool channel
18e: treatment tool lead-out port
19: operation part
19a: angle knob
19b: elevating operation lever
19c: treatment tool lead-in port
19d: air and water supply button
19e: suction button
21: flexible sheath
22: distal end portion
23: operation part
31: distal end portion body
32: cap
32A: peripheral surface portion
32B: end surface portion
32C: opening window
33: elevator
34: elevating operation wire
35: signal cable
36: light guide
37: disk portion
37A: first opening portion
38: partition wall portion
38A: upper end surface
39: partition wall portion
41: elevator housing portion
42: lens cover
43: air and water supply nozzle
44: packing
45: imaging unit
46: imaging unit housing chamber
46A: locking claw
46B: lens cover opening portion
47: image sensor
48: illumination optical system
49: case member
49A: proximal end surface
49B: upper end surface
49C: fitting hole
50: jig
50A: distal end surface
50B through-hole
51: connection piece
52: connection opening portion
61: distal end portion body
62: lens cover
62A: cutout portion
63: packing
64: pressing member
64A: inclined surface
65: imaging unit
66: imaging unit housing chamber
66A: locking claw
66B: lens cover opening portion
66C: cutout portion
66D: corner portion
66E: rail groove
66F: screw hole
66G: cutout portion
67: case member
67A: upper end surface
67B: proximal end surface
67C: chamfer portion
67D: cutout portion
68: jig
69: screw
71: distal end portion body
72: fixing member
72A: male screw portion
72B: slit
73: ring member
73A: female screw portion
74: disk portion
74A: opening portion
74B: engaging claw
75: packing
75A: opening portion
75B: through-hole
76: lens cover
77: packing
78: imaging unit
79: imaging unit housing chamber
79A: locking claw
79B: lens cover opening portion
79C: pressing target piece
80: relay connector
81: unlocking hole
82: jig
90: distal end portion body
91: disk portion
91A: opening portion
91B: slit
92: partition wall portion
92A: opening portion
92B: engaging claw
92C: upper end surface
93: lens cover
94: packing
96: imaging unit housing chamber
96A: locking claw
96B: lens cover opening portion
96C: pressing target piece
97: packing
97A: opening portion
101: unlocking hole
102: jig
B: boundary
L1: long side

What is claimed is:

1. An endoscope comprising:
an insertion part that is to be inserted into a subject;
a distal end portion body that is disposed at a distal end of the insertion part; and
an imaging unit that is housed and attachably and detachably held in the distal end portion body in an airtight or liquidtight state with respect to an external space,
wherein the imaging unit has an image sensor that observes a visual field region in a first direction perpendicular to an axial direction of the insertion part.

2. The endoscope according to claim 1,
wherein the distal end portion body includes:
a lens cover;
a holding portion in which the imaging unit is held; and
a first opening portion that is positioned on a proximal end side of the holding portion, and
the imaging unit is separated from the holding portion through the first opening portion in a case where the imaging unit held in the holding portion is pulled toward a proximal end side of the distal end portion body.

3. The endoscope according to claim 2,
wherein the holding portion includes a locking portion that locks the imaging unit, and
the imaging unit is allowed to move to the proximal end side in a case where the imaging unit is unlocked.

4. The endoscope according to claim 3,
wherein the locking portion is a locking claw that locks a proximal end surface of the imaging unit.

5. The endoscope according to claim 1,
wherein the distal end portion body includes:
a lens cover through which at least a portion of the imaging unit is exposed;
a second opening portion at which the lens cover is attachably and detachably held; and
a holding portion in which the imaging unit is held, and
the imaging unit is separated from the holding portion through the second opening portion in a case where the lens cover held at the second opening portion is removed and the imaging unit held in the holding portion is pulled toward the second opening portion side.

6. The endoscope according to claim 5,
wherein the distal end portion body includes a rail portion that is slidably fitted to a side edge of the lens cover, and
the lens cover is disposed at a position facing the imaging unit in a case where the side edge is fitted to the rail portion.

7. The endoscope according to claim 6,
wherein the distal end portion body includes a retaining member that is attachably and detachably coupled to the distal end portion body on a trajectory of the lens cover sliding with respect to the rail portion, and
the retaining member restricts the lens cover from being separated in the slide direction.

8. The endoscope according to claim 1,
wherein the insertion part is composed of a distal end portion including the distal end portion body, a bendable portion installed consecutively to the distal end portion, and a soft portion installed consecutively to the bendable portion,
the distal end portion body includes:
a lens cover through which at least a portion of the imaging unit is exposed;
a holding portion in which the imaging unit is held; and
an engaging portion that attachably and detachably engages with the bendable portion or a fixing member fixed to the bendable portion, and
the imaging unit is separated, integrally with the holding portion, from the bendable portion or the fixing member in a case where the engaging portion is disengaged from the bendable portion or the fixing member.

9. The endoscope according to claim 8, further comprising:
a packing member that maintains airtightness or liquidtightness between the holding portion and the bendable portion or the fixing member.

10. The endoscope according to claim 2,
wherein the imaging unit includes a chamfer portion that is inclined from an upper end surface facing the lens cover to a proximal end surface or from the upper end surface to a distal end surface,
the distal end portion body includes an elastic body that is disposed between the lens cover and the imaging unit, and
the imaging unit comes into contact with a distal end surface or a proximal end surface of the holding portion or a corner portion of the holding portion that is positioned diagonally opposite to the chamfer portion with the chamfer portion being pressed by an elastic force of the elastic body.

11. The endoscope according to claim 2, further comprising:
a relay connector that is coupled to the imaging unit; and
a cable that is connected to the imaging unit via the relay connector,
wherein the relay connector is disposed inside the distal end portion body.

12. The endoscope according to claim 2,
wherein the lens cover is fixed to the distal end portion body, and
the distal end portion body includes a jig cutout portion that adjoins the lens cover, for insertion of a jig that unfixes the lens cover.

13. The endoscope according to claim 8,
wherein the holding portion includes a locking portion that locks the imaging unit,
the distal end portion body includes an unlocking hole that communicates with the holding portion, and
the imaging unit is unlocked or at least a portion of the locking portion is removed in a case where a jig is inserted through the unlocking hole to press the locking portion.

14. The endoscope according to claim 13,
wherein the locking portion is a locking claw that locks a proximal end surface of the imaging unit.

15. The endoscope according to claim 1,
wherein the imaging unit includes a light source that irradiates a subject with illumination light.

16. The endoscope according to claim 1,
wherein at least a portion of the endoscope is disposable except the imaging unit.
